SEQUENCE LISTING (13 sequences)

SEQ ID NO: 1 - protein, 591 aa, Homo sapiens
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - DNA, 2032 bp, Homo sapiens
tccatattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatg

SEQ ID NO: 3 - protein, 8 aa, Homo sapiens
EMTPVNPG

SEQ ID NO: 4 - protein, 34 aa, Homo sapiens
LSEDKLLACGEGAADIIIGHLCIRHEMTPVNPGV

SEQ ID NO: 5 - protein, 197 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRES

SEQ ID NO: 6 - protein, 192 aa, Homo sapiens
SLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 7 - protein, 201 aa, Homo sapiens
QALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 8 - protein, 389 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 9 - protein, 393 aa, Homo sapiens
SLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 10 - protein, 331 aa, Homo sapiens
QDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 11 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQPNDLEACVMQEERVGETPLMNADSILAVKKYFRRITLYLTEKKYSPCAWEVVRAEIVRSLSLSTNLQERLRRKE

SEQ ID NO: 12 - protein, 166 aa, Homo sapiens [sequence omitted]

SEQ ID NO: 13 - protein, 144 aa, Homo sapiens [sequence omitted]

(19) United States
(12) Patent Application Publication
(10) Pub. No.: US 2010/0028297 A1
(43) Pub. Date: Feb. 4, 2010

(54) COADMINISTRATION OF ALPHA-FETOPROTEIN AND AN IMMUNOMODULATORY AGENT TO TREAT MULTIPLE SCLEROSIS

(76) Inventors: Edward J. Stewart, Winchester, MA (US); Michael Briskin, Lexington, MA (US)

Correspondence Address: CLARK & ELBING LLP, 101 FEDERAL STREET, BOSTON, MA, 02110, US

(21) Appl. No.: 12/520,045

(22) PCT Filed: Dec. 19, 2007
(86) PCT No.: PCT/US07/26015

§ 371 Date: Sep. 30, 2009

Related U.S. Application Data
(60) Provisional application No. 60/876,027, filed on Dec. 19, 2006.

(51) Int. Cl. A61K 38/21; A61K 38/16; A61K 39/395

(52) U.S. Cl. 424/85.4; 514/12; 424/130.1

(57) ABSTRACT

The present invention features methods for treating multiple sclerosis by administering an alpha-fetoprotein polypeptide (or a biologically active fragment, derivative, or analog thereof) and one or more immunomodulatory agents to a patient in need thereof. Also disclosed are compositions and kits that contain an alpha-fetoprotein polypeptide (or a biologically active fragment, derivative, or analog thereof) and one or more immunomodulatory agents.

### FIELD OF THE INVENTION

This invention relates to treatment methods using alpha-fetoprotein, including its functional fragments, analogs, and derivatives, in conjunction with the administration of one or more immunomodulatory agents for the treatment of multiple sclerosis.

### BACKGROUND OF THE INVENTION

Multiple Sclerosis (MS) is a neurological disease characterized by irreversible degeneration of the nerves of the central nervous system (CNS). Although the underlying cause is unclear, the neurodegeneration in MS is the direct result of the demyelination of nerves (i.e., the stripping of myelin, a protein that normally lines the outer layer and insulates the nerves, away from nerves). As the condition progresses, patches of inflammation and scarring develop, which interferes with the function of nerves. Consequently, an MS patient gradually loses sensory and motor functions of the body.

MS can range from relatively benign, to somewhat disabling, to devastating, as communication between the brain and other parts of the body becomes disrupted. Although the exact mechanism of the demyelination is undetermined, many investigators believe MS to be an autoimmune disease—one in which the body, through its immune system, launches a defensive attack against its own tissues. In the case of MS, it is the nerve-insulating myelin that comes under assault. As the myelin gradually degenerates and eventually disappears, the electrical impulses that travel along the nerves decelerate. Late in the disease, the nerves themselves become damaged. As more and more nerves are affected, a patient experiences a progressive interference with functions that are controlled by the nervous system such as vision, speech, walking, writing, and memory. About 250,000 to 350,000 people in the U.S. suffer from MS. Most people experience their first symptoms of MS between the ages of 20 and 40, but MS has been diagnosed as early as age 15 and as late as age 60. MS is progressively destructive unless the patient receives medical therapy that is effective in halting or slowing the deterioration. While some individuals manage well in the short term, MS patients invariably become more significantly impaired by the disease over time.

MS is known to be treated with various therapeutic modalities, including Type I interferon (IFN), such as IFN-β-1a and IFN-β-1b (see, e.g., Goodin, Int. M.S. J., 12(3):96-108, 2005). While generally well-tolerated and effective, interferon therapy can lead to the generation of neutralizing antibodies in a patient, which dramatically diminish the efficacy of the therapy. Thus, there remains a need for new, effective therapeutic approaches for treating MS. The present invention addresses this and other related needs.

### SUMMARY OF THE INVENTION

The invention provides a method of treating a patient with MS by administering to the patient a therapeutically effective amount of an alpha-fetoprotein (AFP) or a biologically active fragment, derivative, or analog thereof, and a therapeutically effective amount of an immunomodulatory agent.

In related embodiments of the above method, the AFP or biologically active fragment, derivative, or analog thereof or the immunomodulatory agent is administered daily, weekly, biweekly, or monthly. In different embodiments of the above method, the AFP or biologically active fragment, derivative, or analog thereof is administered in the range of about 0.5 mg to 400 mg per dose, or the immunomodulatory agent is administered in the range of about 50 μg to 300 mg per dose.

In different embodiments of the method, the AFP or biologically active fragment, derivative, or analog thereof and the immunomodulatory agent are administered coextensively, e.g., in separate dosage forms or in the same dosage form, or is administered separately.

In related embodiments of the method, the AFP or the biologically active fragment, derivative, or analog thereof is administered prior to or after the immunomodulatory agent.

In different embodiments of all the method, the AFP or biologically active fragment, derivative, or analog thereof or the immunomodulatory agent is administered intravenously, intramuscularly, orally, by inhalation, parenterally, intraperitoneally, intraarterially, transdermally, sublingually, nasally, in a suppository, transbuccally, liposomally, adiposally, intraocularly, subcutaneously, intrathecally, topically, or through local administration. In additional embodiments, the AFP (or biologically active fragment, derivative, or analog thereof) and the immunomodulatory agent are administered by two different routes of administration or are administered by the same route of administration.

In related embodiments of the method, one or more secondary agents (e.g., a disease-modifying anti-rheumatic drug (DMARD), a corticosteroid, or a non-steroidal anti-inflammatory drug (NSAID)) is administered to the patient in addition to the AFP (or biologically active derivative, fragment, or analog thereof) and the immunomodulatory agent.

In different embodiments of the method, administration of AFP (or biologically active fragment, derivative, or analog thereof) and an immunomodulatory agent results in a loss of or reduction in the severity of one or more symptoms of MS (e.g., tingling, numbness, tremors, loss of balance, weakness in one or more limbs, blurred or double vision, slurred speech, swallowing problems, paralysis, lack of coordination, cognitive difficulties, fatigue, muscle spasms, dizziness, breathing problems, and seizures; e.g., a reduction of at least 20% in the severity of one or more symptoms of MS).

The invention further provides compositions containing an AFP (or biologically active fragment, derivative, or analog thereof) and an immunomodulatory agent in a therapeutically effective amount to treat multiple sclerosis in a patient. In an embodiment, one or more secondary agents (e.g., a DMARD, corticosteroid, or NSAID) are present in the composition in addition to the AFP (or biologically active fragment, derivative, or analog thereof) and the immunomodulatory agent.

The invention also provides kits containing a therapeutically effective amount of an AFP (or biologically active fragment, derivative, or analog thereof), a therapeutically effective amount of an immunomodulatory agent, and instructions for administering the AFP and the immunomodulatory agent to a patient having multiple sclerosis. In several embodiments of the kits of the invention, the AFP (or biologically active fragment, derivative, or analog thereof) and the immunomodulatory agent are formulated for two different routes of administration or formulated for the same route of administration. In related embodiments, the kit further contains one or more secondary agents (e.g., a DMARD, corticosteroid, or NSAID) for administration to a patient in combination with the AFP and the immunomodulatory agent.

In different embodiments of the above compositions and kits, the AFP (or biologically active fragment, derivative, or analog thereof) and/or the immunomodulatory agent is formulated for intravenous, oral, inhalatory, parenteral, intraperitoneal, intraarterial, transdermal, sublingual, nasal, in a suppository, transbuccal, liposomal, adiposal, intraocular, subcutaneous, intrathecal, topical, or local administration.

In different embodiments of all aspects of the invention, the AFP is human recombinant AFP or non-glycosylated AFP. In additional embodiments of all aspects of the invention, the immunomodulatory agent is a peptide or protein (e.g., interferon-β-1a, interferon-β-1b, interferon-α, interferon-γ, and interferon-τ, and glatiramer acetate (Copaxone®)), an antibody (e.g., nataluzinab, daclizumab, rituximab, ABT-874, and alemtuzumab), small molecule (e.g., BG 12 (fumarate), fingolimod (FTY-720), mixoxantrone (Novantrone®), laquinimod, teriflunomide, and atorvastatin), or one of the agents listed in FIG. 5.

### DEFINITIONS

In this application, when two therapeutic agents are “administered coextensively,” the administration time periods of the agents may completely overlap or at least in part overlap. When the administration of the two agents is not coextensive, the two therapeutic agents are preferably administered in time periods that do not overlap; the administration preferably occurs within the bioactive period of one of the two therapeutic agents, i.e., the earlier administered agent retains at least a substantial portion of its biological activity in the patient at the time when the latter administered agent is delivered. In other cases where two therapeutic agents are not administered coextensively, one agent may be administered outside of the other agent's bioactive period.

As used herein, the term “alpha-fetoprotein” or “AFP” refers to a polypeptide having an amino acid sequence substantially identical to the mature human AFP (SEQ ID NO: 1) or a nucleic acid that encodes the polypeptide (NCBI Accession No. NM_{—}001134; SEQ ID NO: 2). Mature human AFP is a protein of 591 amino acids (see SEQ ID NO:1), resulting from cleavage of a precursor of 609 amino acids (GenBank Accession No. NP_{—}001125) to remove an 18-amino acid signal sequence. An AFP of this invention has an amino acid sequence that is substantially identical to SEQ ID NO: 1. The AFP is not limited to the full-length sequence; it also includes biologically active fragments of AFP. An AFP of the invention also includes any recombinant human AFP (whether or not having the same post-translational modifications as the naturally occurring version) and biologically active variants of human AFP (e.g., a non-glycosylated form of AFP, see, e.g., U.S. Pat. No. 7,208,576).

In some embodiments, the AFP of this invention may contain modifications of the amino acid sequence of SEQ ID NO: 1, including substitution (e.g., conservative substitution), deletion, or addition of some amino acid residues. For instance, a recombinant human AFP is described in U.S. Pat. No. 7,208,576, incorporated herein by reference, which contains an asparagine to glutamine substitution at position 233 of SEQ ID NO: 1. The term “alpha-fetoprotein” also encompasses any derivatives or analogues of AFP described herein.

An AFP of this invention has the same or substantially the same biological activity (e.g., at least 50%, preferably at least 60%, 70%, or 80%, and more preferably at least 90%, 95%, or 99% or more) as the native human AFP. For example, an AFP of the invention, like native human AFP, exhibits the ability to bind to human leukocytes and the ability to suppress autoimmune reactions. The leukocyte binding assay used for testing AFP activity is described herein and in, e.g., Parker et al., Protein Express. Purification 38:177-183, 2004. The desired autoimmune suppression activity for an AFP of this invention can be demonstrated by assaying the ability of the AFP to suppress human autologous mixed lymphocyte reactions (AMLR) or by assaying the ability of the AFP to suppress experimental autoimmune encephalomyelitis (EAE) in a mouse model. Such activity can be verified by assays described herein. A functional AFP of the invention demonstrates at least 40%, 50%, 60%, 70%, 80%, 85%, 90%, 95%, or 100% of the ability of the native human AFP to bind human monocytes in an assay described in Parker et al., supra, and at least 40%, 50%, 60%, 70%, 80%, 85%, 90%, 95%, or 100% of the ability of the native human AFP to suppress autoimmune reactions. The latter activity is shown by suppression of human AMLR in an assay described in U.S. Pat. No. 5,965,528, or, in the alternative, by suppression of the development of EAE in a mouse model (see, e.g., Fritz et al., J. Immunol. 130:1024, 1983; Naiki et al., Int. J. Immunopharmacol. 13:235,1991; and Goverman, Lab. Anim. Sci., 46:482, 1996).

An AFP fragment of the invention can be determined using one or more assays described herein (e.g., AMLR assays, AFP-binding to monocyte assays, experiments using the EAE mouse model, and splenocyte assays). A typical biologically active AFP fragment contains at least 5 contiguous amino acids of SEQ ID NO: 1, or at least 8 contiguous amino acids, preferably at least 10, 20, or 50 contiguous amino acids, more preferably at least 100 contiguous amino acids, and most preferably at least 200, 300, 400, or more contiguous amino acids in length. For instance, U.S. Pat. No. 6,818,741 discloses an 8-amino acid fragment of human AFP (amino acids 471-478; EMTPVNPG; SEQ ID NO: 3) as well as other AFP fragments containing this 8-mer. An active AFP fragment of this invention may further contain amino acid substitution, deletion, or addition at a limited number of positions, so long as the AFP fragment has at least 90% identity to its corresponding sequence within SEQ ID NO: 1. For sequence comparison purposes in this application, the corresponding sequence of SEQ ID NO: 1 is deemed to have the same number of amino acids as a given AFP fragment. For instance, a 34-mer AFP peptide corresponding to the 446-479 segment of SEQ ID NO: 1 (LSEDKLLACGEGAADIIIGHLCIRHEMTPVNPGV; SEQ ID NO: 4) may contain up to 3 amino acids altered from the 446-479 segment of SEQ ID NO: 1. One such example of sequence deviation in biologically active AFP fragments is found in U.S. Patent No. 5,707,963, which discloses a 34-amino acid fragment of human AFP (SEQ ID NO: 4) with flexibility at two amino acid residues (amino acid 9 and 22 of SEQ ID NO: 4). Some other examples of AFP fragments include Domain I (amino acids 2-198 of mature human AFP; SEQ ID NO: 5), Domain II (amino acids 199-390 of mature human AFP; SEQ ID NO: 6), Domain III (amino acids 391-591 of mature human AFP; SEQ ID NO: 7), Domain I+II (amino acids 2-390 of mature human AFP; SEQ ID NO: 8), Domain II+III (amino acids 199-591 of mature human AFP; SEQ ID NO: 9), and human AFP Fragment I (amino acids 267-591 of mature human AFP; SEQ ID NO: 10).

In this application, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones (e.g., peptide mimetics, such as an AFP peptoid), but retain the same basic chemical structure as a naturally occurring amino acid. Amino acid mimetics refers to chemical compounds that have a structure different from the general chemical structure of an amino acid, but capable of functioning in a manner similar to a naturally occurring amino acid.

As to amino acid sequences, one of skill will recognize that individual substitutions, deletions, or additions to a polypeptide sequence that alter, add, or delete a single amino acid or a small percentage of amino acids in the sequence constitute a “conservatively modified variant,” when the alterations result in the substitution of one or more amino acids with other, chemically similar amino acids. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

By the term “biologically active” is meant having one or more activities known to be associated with a naturally occurring or synthetic peptide, polypeptide, protein, antibody, compound, small molecule, or fragment, derivative, or analog thereof (e.g., an AFP or an immunomodulatory agent).

The term “disease modifying anti-rheumatic drug” or “DMARD” refers to a therapeutic agent that is used for the treatment of an inflammatory disease. A DMARD can be used treat, prevent, or reduce one or more of the symptoms of or the progression of an inflammatory disease in a patient when administered in a therapeutically effective amount. Examples of DMARDs known in the art include auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

By “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

As used here, “immunomodulatory agent” refers to (1) an interferon or a peptide or protein that has an amino acid sequence substantially identical (e.g., at least 70%, 75%, 80%, 85%, 90%, 95%, 96%, 97%, 98%, 99%, or even 100% identical) to all or a portion of the sequence of an interferon (e.g., a human interferon), such as IFN-α (e.g., IFN-α-1a; see U.S. Patent Application No. 20070274950, incorporated herein by reference in its entirety), IFN-α-1b (SEQ ID NO: 11), IFN-α-2a (see PCT Application No. WO 07/044083, herein incorporated by reference in its entirety) and IFN-α-2b (SEQ ID NO: 12)), IFN-β (e.g., described in U.S. Pat. No. 7,238,344, incorporated by reference in its entirety; IFN-β-1a (as described in U.S. Pat. No. 6,962,978; incorporated by reference in its entirety) and IFN-β-1b (as described in U.S. Pat. Nos. 4,588,585; 4,959,314; 4,737,462; and 4,450,103; incorporated by reference in their entirety), IFN-γ (e.g., SEQ ID NO: 13), and IFN-τ (as described in U.S. Pat. No. 5,738,845 and U.S. Patent Application Publication Nos. 20040247565 and 20070243163; incorporated by reference in their entirety), or a peptide, such as glatiramer acetate (Copaxone®); (2) a small molecule (e.g., BG12 (fumarate), fingolimod (FTY-720), laquinimod, teriflunomide, or atorvastatin, or a molecule that demonstrates the same or substantially the same biological activity to an interferon (e.g., at least 50%, 60%, 70%, 75%, 80%, 85%, 90%, 95%, or 100% of the activity of a human IFN-α, a human IFN-β, a human IFN-γ, or a human IFN-τ in the ability to suppress EAE in a mouse model)); (3) an antibody (e.g., all or part of a monoclonal antibody (e.g., an α4 integrin-binding antibody, such as nataluzimab; an IL-2 receptor-binding antibody, such as daclizumab; a CD20-binding antibody, such as rituximab; an IL-12 binding antibody, such as ABT-874; and a CD52-binding antibody, such as alemtuzumab), a polyclonal antibody, or an antibody fusion protein); (4) a peptide (e.g., MBP-8289, NBI-5788, and T cell receptor peptide (Neurovax®)); or (5) a DNA vaccine (e.g., BNT-3009-01). Non-limiting exemplary immunomodulatory agents have the ability to decrease (e.g., by at least 10%, 15%, 20%, 25%, 30%, 35%, 40%, 45%, 50%, 60%, 70%, 80%, 90%, or even 100%) the severity of one or more symptoms of MS, or the ability to prevent, inhibit, or decrease (e.g., by at least 10%, 15%, 20%, 25%, 30%, 35%, 40%, 45%, 50%, 60%, 70%, 80%, 90%, or even 100%) the progression of MS in a patient (e.g., demyelination of nerves and the frequency or severity of one or more symptoms of MS).

Desirable immunomodulatory agents are proteins that have at least 50% (more preferably at least 70%, 75%, 80%, 85%, 90%, 95%, 96%, 97%, 98%, 99%, or even 100%) amino acid sequence identity to a human IFN-α, -β, -γ, or -τ and have at least 50% (more preferably at least 50%, 60%, 70%, 75%, 80%, 85%, 90%, 95%, or 100%) activity of human IFN-β-1a in the ability to suppress EAE in a mouse model, similar to the assay system used for verifying AFP activity. An immunomodulatory agent within the meaning of this invention encompasses both naturally occurring interferons as well as recombinantly produced interferons. A recombinantly produced interferon may contain modification to one or more amino acid residues, including deletion, addition, and substitution, or it may contain a different pattern of post-translational modification (e.g., glycosylation, PEGylation, and the like). Non-limiting exemplary immunomodulatory agents suitable for use with the present invention include Rebif® (IFN-β-1a), Avonex® (IFN-β-1a), Betaseron® (IFN-β-1b), Tauferon™ (IFN-τ), Roferon-A® (IFN-α-2a), Intron-A® (IFN-α-2b), Rebetron® (IFN-α-2b), Alferon-N® (IFN-α-n3), Peg-Intron® (IFN-α-2b covalently conjugated with monomethoxy polyethylene glycol), Infergen® (a non-naturally occurring type 1 interferon with 88% homology to IFN-α-2b), Actimmune® (IFN-γ-1b), Pegasys® (pegylated IFN-α-1a), Copaxone® (glatiramer acetate), and Novantron® (mixoxantrone). Additional examples of immunomodulatory agents are listed in FIG. 5; one or more of these immunomodulatory agents can be combined with an AFP to produce a composition of the invention for use in the methods of treating MS, as described herein.

The term “multiple sclerosis” or “MS” refers to a disease in which the nerves of the central nervous system (brain and spinal cord) degenerate as the result of demyelination. The protein myelin normally provides a covering or insulation for nerves.

By “non-steroidal anti-inflammatory drug” or “NSAID” is meant a non-steroidal agent that prevents or diminishes inflammation. Examples of NSAIDs include naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, tolmetin, and COX-2 inhibitors such as rofecoxib, celecoxib, valdecoxib, or lumiracoxib.

The term “substantial identity” or “substantially identical,” when used in the context of comparing a polynucleotide or polypeptide sequence to a reference sequence, refers to the fact that the polynucleotide or polypeptide sequence is the same as the reference sequence or has a specified percentage of nucleotides or amino acid residues that are the same at the corresponding locations within the reference sequence when the two sequences are optimally aligned. For instance, an amino acid sequence that is “substantially identical” to a reference sequence has at least about 60% identity, preferably 65%, 70%, 75%, 80%, 85%, 90%, 91%, 92%, 93%, 94%, 95%, 96%, 97%, 98%, 99%, or higher percentage identity (up to 100%) to the reference sequence (e.g., the mature human AFP amino acid sequence as set forth in SEQ ID NO:1, or a fragment thereof, or an interferon), when compared and aligned for maximum correspondence over the full length of the reference sequence as measured using a BLAST or BLAST 2.0 sequence comparison algorithms with default parameters, or by manual alignment and visual inspection (see, e.g., NCBI web site).

By “synergistic effect” is meant that the administration of alpha-fetoprotein (or a biologically active fragment, derivative, or analog thereof) and one or more immunomodulatory agents in a therapeutically effective amount for the treatment of MS exhibits an additive therapeutic effect (e.g., an at least 1%, 5%, 10%, 15%, 20%, 25%, 30%, 35%, 40%, 45%, 50%, 60%, or 70% or greater improvement in the resolution of MS or an at least 1%, 5%,10%,15%, 20%, 25%, 30%, 35%, 40%,45%, 50%, 60%, or 70% decrease in the severity or frequency of one or more symptoms of MS; see, “treating”), which is greater than that observed when the AFP or the one or more immunomodulatory agents are administered alone. A synergistic effect can also mean that the administration of AFP (or biologically active fragment, derivative, or analog) and one or more immunomodulatory agents in combination allows either the AFP, the one or more immunomodulatory agents, or both to be administered at a lower dosage than that normally required for achieving the same or a substantially similar result in therapy as compared to the amount of the AFP or the one or more immunomodulatory agents needed when administered alone (e.g., an at least 10%, 15%, 20%, 25%, 30%, 35%, 40%, 45%, 50%, 60%, or 90% lower dosage of the AFP, one or more immunomodulatory agents, or both). A synergistic effect between an AFP (or biologically active fragment, derivative, or analog thereof) and one or more immunomodulatory agents may also be observed in instances where the toxicity of the one or more immunomodulatory agents is decreased (e.g., by at least 5%, 10%, 15%, 20%, 25%, 30%, 35%,40%,45%, 50%, 60%, 70% or more) when administered in conjunction with the AFP relative to the toxicity of the one or more immunomodulatory agents when administered at the same concentration in the absence of the AFP. A synergistic effect may also occur upon co-administration of AFP (or biologically active fragment, derivative, or analog thereof) and one or more immunomodulatory agents, in which the co-administration allows for an increase in the dosage (e.g., by at least 5%,10%,15%, 20%, 25%, 30%, 35%,40%,45%, 50%, 55%, 60%, 65%, 70%, 75%, 85%, 90%, 95%, 100% or more) of the one or more immunomodulatory agents beyond that normally administered for treating MS without the toxicity normally expected or observed at the increased dose of the one or more immunomodulatory agents.

By “treating” is meant the reduction (e.g., by at least 10%, 15%, 20%, 25%, 30%, 35%, 40%, 45%, 50%, 60%, 70%, 80%, 90%, 95%, 99%, or even 100%) in the progression, severity, or frequency of one or more symptoms of MS (e.g., tingling, numbness, tremors, loss of balance, weakness in one or more limbs, blurred or double vision, slurred speech, swallowing problems, paralysis, lack of coordination, cognitive difficulties (e.g., decreased memory and concentration), fatigue, muscle spasms, dizziness, breathing problems, and seizures) or the prevention or decrease (e.g., by at least 10%, 15%,20%,25%,30%, 35%,40%,45%, 50%, 60%, 70%, 80%, 90%, 95%, 99%, or even 100%) in the progression of MS in a human patient (e.g., demyelination of nerves and the frequency or severity of one or more symptoms of MS).

A “therapeutically effective amount” of a therapeutic agent (e.g., an AFP, immunomodulatory agent, DMARD, corticosteroid, NSAID, or other agent of the invention) is an amount of the agent that is sufficient to effectuate a desired therapeutic effect on a given condition or disease. The amount may vary depending on the effect to be achieved. For instance, a “therapeutically effective amount” of an immunomodulatory agent for treating MS alone may be different from the “therapeutically effective amount” of an immunomodulatory agent used to treat MS in combination with AFP (or a biologically active fragment, derivative, or analog thereof; e.g., the therapeutically effective amount of the immunomodulatory agent may be reduced when administered in combination with an AFP).

### BRIEF DESCRIPTION OF THE FIGURES

FIG. 1 shows the amino acid (SEQ ID NO: 1) of mature human AFP and the mRNA nucleic acid sequence of human AFP (SEQ ID NO: 2). N indicates the asparagine 233 glycosylation site in the mature human AFP amino acid sequence.

FIG. 2 shows the amino acid sequences of biologically active fragments of AFP including amino acids 2-198 (Domain I; SEQ ID NO: 5), amino acids 199-390 (Domain II; SEQ ID NO: 6), amino acids 391-591 (Domain III; SEQ ID NO: 7), amino acids 2-390 (Domains I+II; SEQ ID NO: 8), amino acids 199-591 (Domains II+III; SEQ ID NO: 9), and amino acids 261-591 of mature human AFP (Human AFP Fragment 1; SEQ ID NO: 10).

FIG. 3 shows the amino acid sequence of human IFN-α-1b (SEQ ID NO: 11) and IFN-α-2b (SEQ ID NO: 12).

FIG. 4 shows the amino acid sequence of human IFN-γ (SEQ ID NO: 13).

FIG. 5 is a table of immunomodulatory agents.

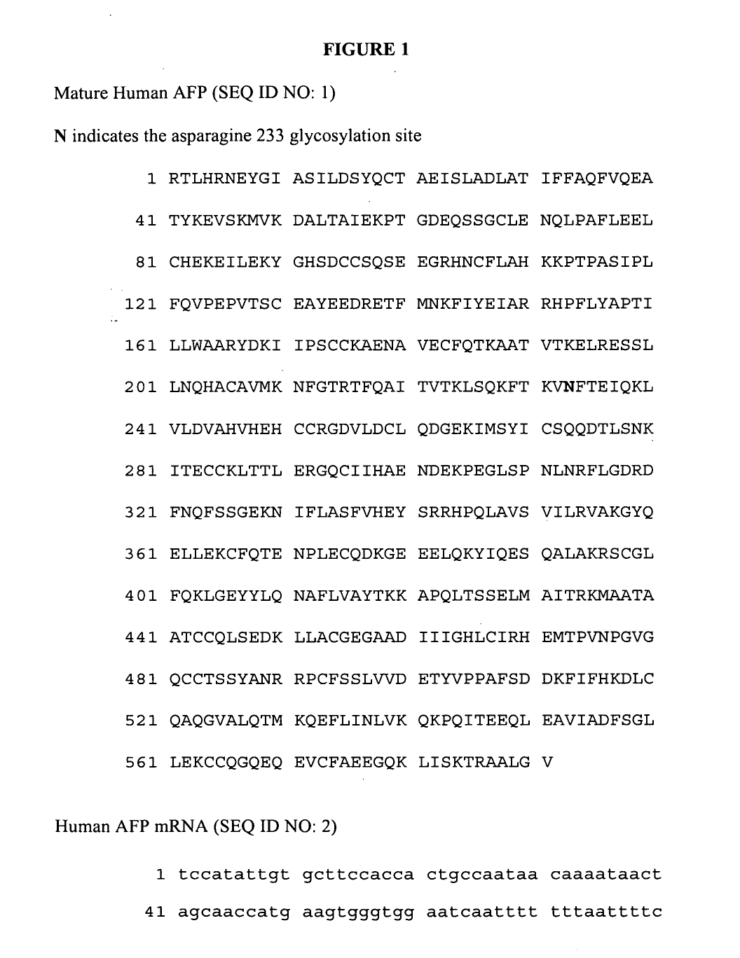

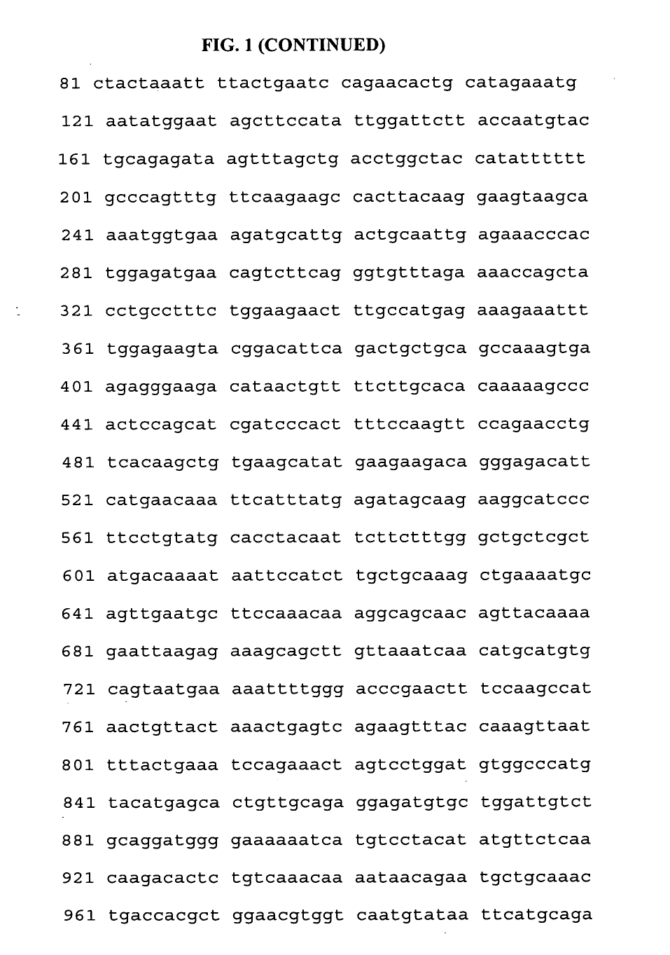

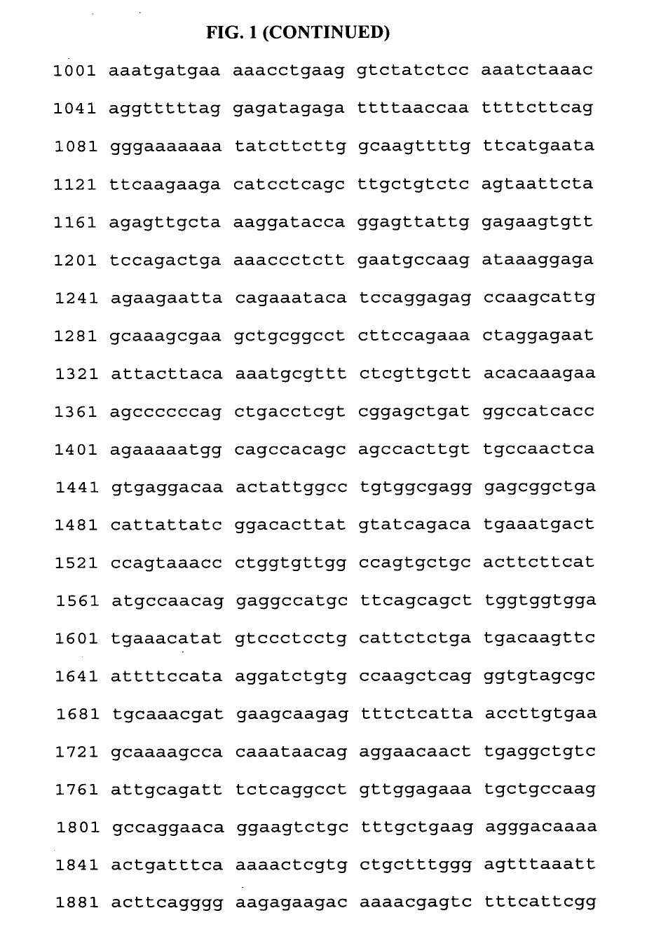

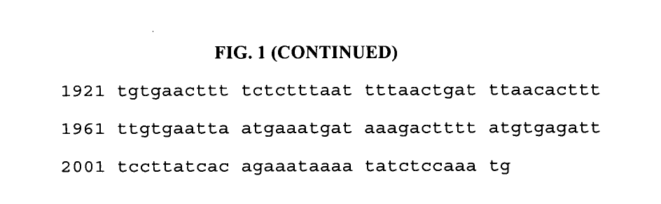

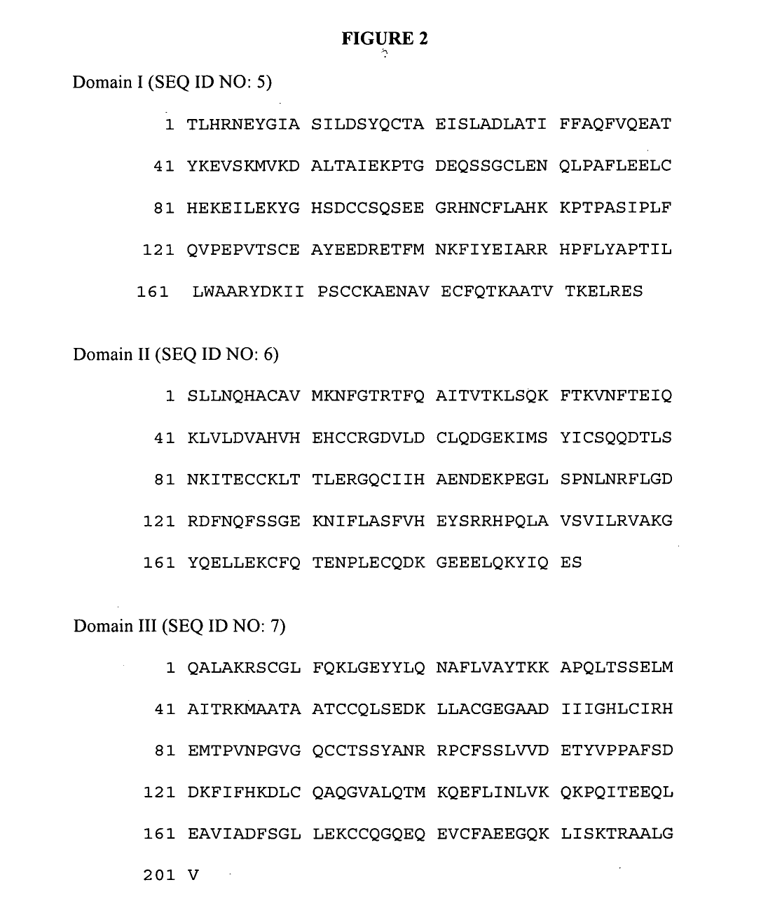

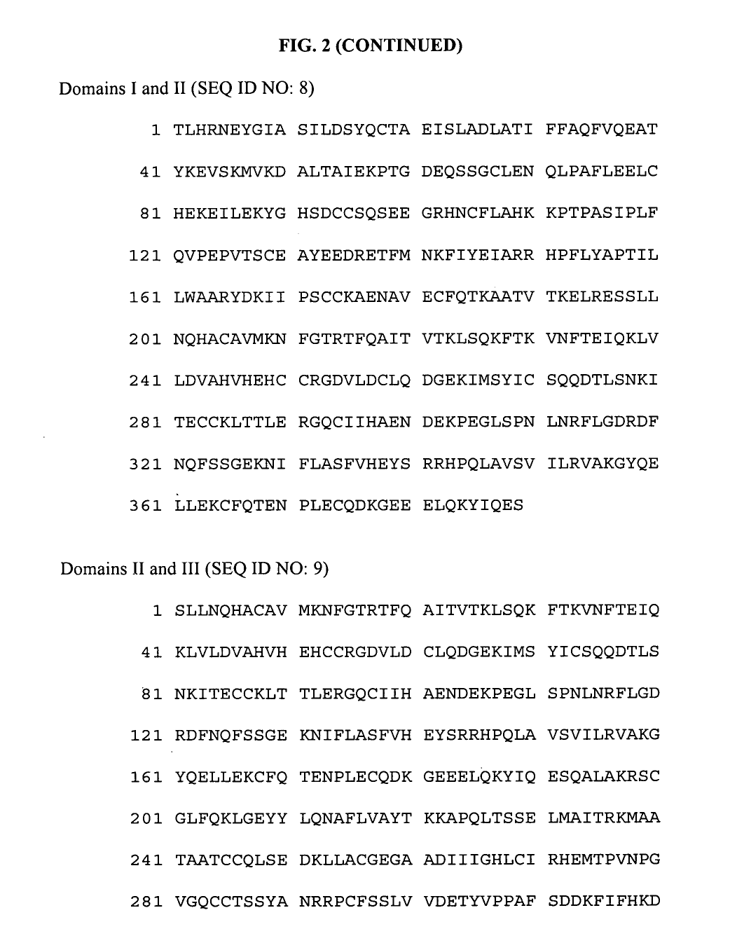

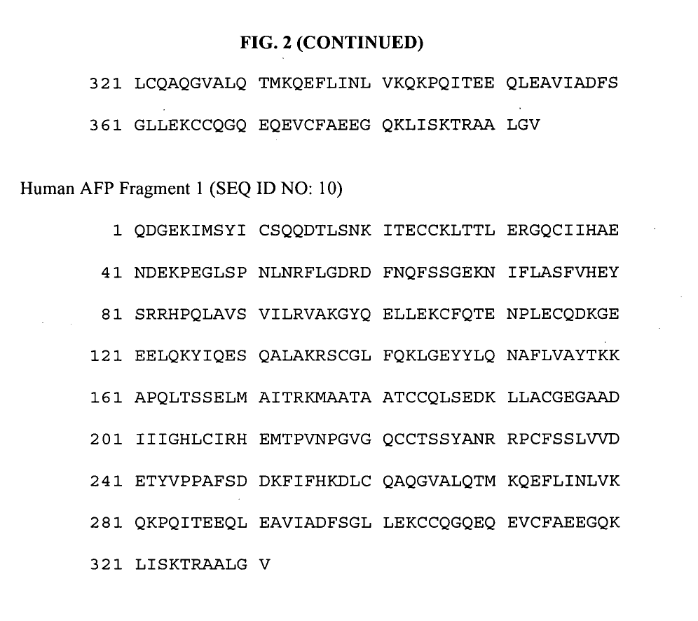

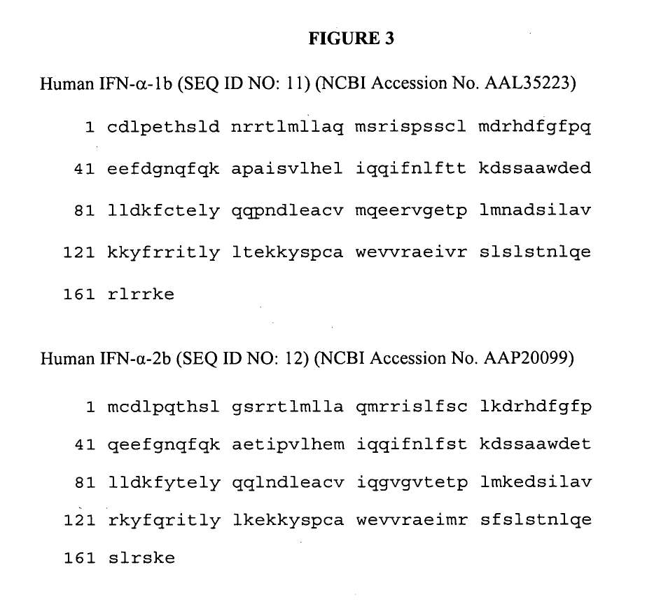

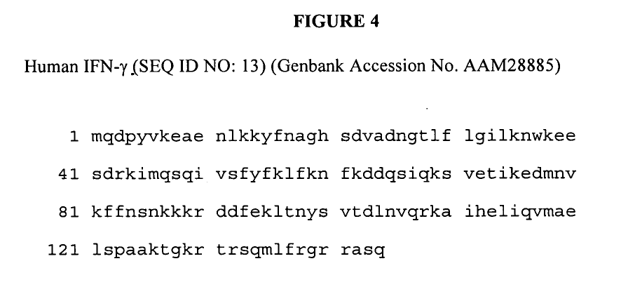

### DETAILED DESCRIPTION

This invention provides a combination treatment for MS. This combination treatment involves co-administering one or more immunomodulatory agents with AFP (or a biologically active fragment, derivative, or analog thereof), each in a therapeutically effective amount, to a patient in need thereof. In another aspect, the invention provides a pharmaceutical composition that includes one or more immunomodulatory agents and AFP (or a biologically active fragment, derivative, or analog thereof), each in a therapeutically effective amount for treating MS. Such a composition optionally includes one or more pharmaceutically acceptable excipients and is formulated to be administered intravenously, intramuscularly, orally, by inhalation, parenterally, intraperitoneally, intraarterially, transdermally, sublingually, nasally, through use of suppositories, transbuccally, liposomally, adiposally, intraocularly, subcutaneously, intrathecally, topically, or through local administration. In a further aspect, the invention provides a kit for treating MS, which includes a therapeutically effective amount of an immunomodulatory agent and AFP (or a biologically active fragment, derivative, or analog thereof), along with proper instructions for the administration of the immunomodulatory agent and AFP (or biologically active fragment, derivative, or analog thereof) to a patient.

### Diagnosis and Monitoring of Multiple Sclerosis

MS can be diagnosed by observing one or more symptoms in a patient. Symptoms of MS may be single or multiple and may range from mild to severe in intensity and short to long in duration. Complete or partial remission from symptoms occurs early in about 70% of MS patients. Visual disturbances often are the first symptoms of MS, but they usually subside. A patient may notice blurred or double vision, red-green distortion, or sudden blindness. Muscle weakness leading to difficulties with coordination and balance commonly is noticed early. Muscle spasms, fatigue, numbness, and prickling pain are common symptoms. There may be a loss of sensation, speech impediment, tremors, dizziness, or occasionally hearing loss. Fifty percent of patients experience mental changes such as decreased concentration, attention deficits, some degree of memory loss, or impairment in judgment. Other symptoms may include depression, manic depression, paranoia, or an uncontrollable urge to laugh and weep called laughing-weeping syndrome. As the disease worsens, patients may experience sexual dysfunction or reduced bowel and bladder control. Heat appears to intensify MS symptoms for about 60% of patients, and relief is found with cold baths or swimming. Pregnancy seems to reduce the number of attacks.

There is no single test for MS. Physicians, particularly neurologists, can take into consideration detailed medical histories and can perform complete physical and neurological examinations in order to diagnose MS. Testing for MS can include, e.g., magnetic resonance imaging (MRI) with intravenous gadolinium or magnetic resonance scanning (MRS), both of which help to identify, describe, and date lesions in the brain (i.e., plaques) that occur in MS patients. Another electro-physiological test, evoked potentials, examines the impulses traveling through the nerves to determine if the impulses are moving normally or too slowly; slower than normal movement of impulses through the nerves is indicative of MS. Finally, examination of the cerebro-spinal fluid that surrounds the spinal cord may be used to identify abnormal chemicals or cells floating in the brain or spinal cord that suggest the presence of MS. Collectively, these three tests strengthen the diagnosis of MS. MS can also be diagnosed by identifying one or more of the following symptoms in a patient: tingling, numbness, tremors, loss of balance, weakness in one or more limbs, blurred or double vision, slurred speech, swallowing problems, paralysis, lack of coordination, cognitive difficulties (e.g., decreased memory and concentration), fatigue, muscle spasms, dizziness, breathing problems, and seizures.

All of the methodologies described above are also useful for monitoring the progression of MS in patients, as well as for monitoring the resolution of MS following treatment using the compositions and methods of the invention (e.g., the resolution or decrease in the severity or frequency of one or more symptoms of MS, such that the effectiveness of the treatment received by the patient can be assessed. In addition, a patient can be assessed for an improvement in MS following treatment (e.g., an improvement in one or more symptoms of MS or in function) using one of several methods known in the art (see, e.g., the Expanded Disability Status Scale (EDSS), Kurtzke, Neurology 33:1444-1452, 1983; and the Multiple Sclerosis Severity Score (MSSS), Roxburgh et al., Neurology 64:1144-1151, 2005. An improvement in one or more of these symptoms (e.g., a decrease in the occurrence, length, or severity of one or more of the symptoms of MS) indicates a therapeutic effect by the compositions and methods of the invention.

### Methods of Treatment of MS by Administration of the Compositions of the Present Invention

The present invention provides methods of treating MS in a patient by co-administering a therapeutically effective amount of an AFP (or biologically active fragment, derivative, or analog thereof) and one or more immunomodulatory agents; the compositions of the invention may, but need not, also include additional therapeutic agents, such as those described below. The compositions of the invention can be administered to a patient to treat, prevent, ameliorate, inhibit the progression of, or reduce the severity of one or more symptoms of MS in a human patient. The AFP (or biologically active fragment, derivative, or analog thereof) and one or more immunomodulatory agents may be administered coextensively or separately, in a single dose or multiple doses. The AFP (or biologically active fragment, derivative, or analog thereof) and one or more immunomodulatory agents may be formulated for the same route of administration or formulated for different routes of administration.

Examples of the symptoms of MS that can be treated using the compositions of the invention include: tingling, numbness, tremors, loss of balance, weakness in one or more limbs, blurred or double vision, slurred speech, swallowing problems, paralysis, lack of coordination, cognitive difficulties (e.g., decreased memory and concentration), fatigue, muscle spasms, dizziness, breathing problems, and seizures. These symptoms of MS, and their resolution during treatment, may be measured by a physician during a physical examination. Additional tests used for the diagnosis of MS or a determination of the severity of MS are described above.

A physician may adjust the dose (e.g., increase or decrease the dose) of the AFP (or biologically active fragment, derivative, or analog thereof) and/or one or more immunomodulatory agents administered to the patient based on the severity of, occurrence of, or progression of, MS in the patient (e.g., based on the severity of one or more symptoms of MS).

The combination therapies of the present invention preferably exhibit a synergistic effect when administered to an MS patient.

### Compositions of the Present Invention

The present invention provides compositions including an AFP (or a biologically active fragment, derivative, or analog thereof) and one or more immunomodulatory agents for the treatment of MS. The compositions of the invention may be formulated for any route of administration (e.g., the formulations described herein) and may be administered in a single dose or multiple doses to a subject in need thereof. The compositions of the invention may also further include secondary agents, e.g., one or more of a DMARD, NSAID, or corticosteroid, as is discussed below.

### AFPs for Use in the Compositions and Methods of the Invention

Alpha-fetoprotein (or a biologically active fragment, derivative, or analog thereof) can be used in the combination treatment method of the present invention. For the purpose of the present invention, both naturally occurring human AFP and recombinantly produced AFP polypeptides (including an active AFP fragment) can be administered. The naturally occurring human AFP can be obtained through purification from, e.g., umbilical cords or umbilical cord serum; whereas a recombinant AFP polypeptide or fragment can be obtained through a prokaryotic or eukaryotic expression system, such as those described in, e.g., U.S. Pat. No. 5,384,250 and U.S. Pat. No. 7,208,576. Using different expression systems may result in a difference in post-translational modification of the recombinant protein. For instance, naturally occurring human AFP is a variably glycosylated protein. In contrast, the recombinant AFP may be unglycosylated when produced by a prokaryotic host cell or may be somewhat differently glycosylated when produced by a eukaryotic host cell. Alternatively, a recombinant AFP can be genetically modified to eliminate glycosylation (e.g., by eliminating a glycosylation site), regardless of the expression system in which it is produced. Human AFP is available through various commercial suppliers, including Fitzgerald Industries International (Concord, Mass.), Cell Sciences (Canton, Mass.), and Biodesign International (Saco, Me.).

Furthermore, it is possible to employ well-known chemical synthesis methods to synthesize an AFP polypeptide or fragment, particularly if the AFP fragment is a peptide of a relatively short length, e.g., a peptide with fewer than 100 or 50 amino acids.

Any AFP polypeptide, regardless of its origin or the presence or absence of post-translational modification(s), can be used in the present invention so long as the polypeptide has the same or substantially the same biological activity relative to naturally occurring AFP (e.g., at least 40%, preferably at least 50%, 55%, 65%, 70%, and 75%, and more preferably at least 80%, 85%, 90%, 95%, 99%, or 100% or more of the biological activity of naturally occurring AFP). Biological activity of an AFP of the invention can be assessed by using one or more of the assays described in more detail below.

Similarly, fragments of human AFP can be used in the composition and treatment method of the present invention, so long as the fragments retain substantially the same biological activity of the naturally occurring human AFP (e.g., as determined using one or more of the assays described herein). Fragments of human AFP can be generated by methods known to those skilled in the art, e.g., proteolytic cleavage or recombinant expression, or may result from normal protein processing (e.g., removal from a nascent polypeptide of amino acids that are not required for biological activity). Chemical methods can also be useful for synthesizing active AFP fragments.

Recombinant human AFP fragments suitable for use in practicing the present invention include Domain I (amino acids 2 (Thr)—198 (Ser) of SEQ ID NO:1 (SEQ ID NO: 5)), Domain II (amino acids 199 (Ser)—390 (Ser) of SEQ ID NO:1 (SEQ ID NO: 6)), Domain III (amino acids 391 (Gln)—591 (Val) of SEQ ID NO:1 (SEQ ID NO: 7)), Domain I+II (amino acids 2 (Thr)—390 (Ser) of SEQ ID NO:1 (SEQ ID NO: 8)), Domain II+III (amino acids 199 (Ser)—591 (Val) of SEQ ID NO:1 (SEQ ID NO: 9)), and rHuAFP Fragment I (amino acids 267 (Met)—591 (Val) of SEQ ID NO:1 (SEQ ID NO: 10)). Other examples of known AFP fragments can be found in, e.g., U.S. Pat. No. 5,707,963 and U.S. Pat. No. 6,818,741, herein incorporated by reference in their entirety.

Also envisioned is the use of functional derivatives or analogs of full-length human AFP or fragments thereof. As described above, such derivatives or analogs can differ from the full-length native human AFP or portions thereof by amino acid sequence differences (e.g., additions, deletions, conservative or non-conservative substitutions), or by modifications (e.g., post-translational modifications) that do not affect sequence, or by both. The derivatives/analogs of the invention will generally exhibit at least 90%, more preferably at least 95%, or even 99% amino acid identity with all or part of the native human AFP amino acid sequence (SEQ ID NO:1).

An AFP derivative/analog may differ from a naturally occurring human AFP due to post-translational modifications (which do not normally alter primary sequence), which include in vivo, or in vitro chemical derivatization of polypeptides, e.g., acetylation, or carboxylation; such modifications may occur during polypeptide synthesis or processing or following treatment with isolated modifying enzymes. Also included are cyclized peptide molecules and analogs that contain residues other than L-amino acids, e.g., D-amino acids or non-naturally occurring or synthetic amino acids, e.g., β or γ amino acids, or L-amino acids with non-natural side chains (see e.g., Noren et al., Science 244:182, 1989). Methods for site-specific incorporation of non-natural amino acids into the protein backbone of proteins is described, e.g., in Ellman et al., Science 255:197, 1992. Also included are chemically synthesized polypeptides or peptides with modified peptide bonds (e.g., non-peptide bonds as described in U.S. Pat. No. 4,897,445 and U.S. Pat. No. 5,059,653) or modified side chains to obtain the desired pharmaceutical properties as described herein. Useful derivatives and analogs are identified for biological activity using art-recognized methods, e.g., those described herein.

### AFP Activity Assays

As stated above, AFP polypeptides, and fragments, derivatives, and analogs thereof, suitable for use in the compositions and methods of the present invention include those that retain the same or substantially the same biological activity as naturally-occurring AFP.

A first assay of AFP polypeptide or fragment, derivative, or analog activity is the measurement of its ability to specifically bind to cellular receptors on human peripheral monocytes. A binding assay suitable for this purpose is described in Parker et al., Protein Express. Purification 38:177-183, 2004. Briefly, a competitive assay format is used to test a candidate AFP polypeptide, fragment, derivative, or analog for its ability to specifically bind to U937 cells, a human monocytic cell line. The cells are maintained in RPMI media with 10% fetal bovine serum. Prior to the binding assay, the cells are washed twice with serum-free media and adjusted to 2.5×10⁶ cells/ml in phosphate-buffered saline (PBS). Native human AFP (SEQ ID NO: 1) or non-glycosylated human AFP (see, e.g., SEQ ID NO: 12, where, e.g., residue 233 is glutamine) is labeled with a detectable label, e.g., fluorescein, in a proper reaction followed by removal of the unattached labeling material, for instance, by gel filtration. In the case of labeling human AFP with fluorescein, the protein is mixed with a solution of fluorescein-5-isothiocyanate in dimethyl sulfoxide for 1 hour in the dark, followed by gel filtration to remove unbound dye. Labeled human AFP is stored in 20% glycerol at −20° C. until use. For the binding assay, a certain number of U937 cells (e.g., 40 μl of cell suspension at 2.5×10⁶ cells/ml concentration) are mixed with a pre-determined amount of labeled human AFP (e.g., at a final concentration of 0.5 μM) with unlabeled human AFP or unlabeled candidate AFP polypeptide or fragment, derivative, or analog, each at a set of final concentrations (e.g., 20, 10, 5, 2.5, 1.25, and 0.625 μM) to determine the IC₅₀ values for both human AFP and the candidate AFP polypeptide or fragment, derivative, or analog. At the conclusion of the binding process, the cells are then washed with PBS and suspended in fresh PBS so that the labeled AFP remaining on U937 cells can be measured, e.g., by flow cytometry.

A second assay of AFP polypeptide or fragment, derivative, or analog activity is the measurement of its ability to suppress autoimmune reactions, either in AMLR or in a mouse model of EAE. Methods are known in the art for testing AMLR and its inhibition. For instance, U.S. Pat. Nos. 5,965,528 and 6,288,034 describe the AMLR system as follows: isolation of human peripheral blood mononuclear cells (PBMC), their fractionation into non-T-cell populations, and the AMLR, performed according to standard procedures. Briefly, responder T-cells are isolated by passing 1.5×10⁸ PMBC over a commercial anti-Ig affinity column (US Biotek Laboratories, Seattle, Wash.) and 2×10⁵ responder cells are subsequently cultured with 2×10⁵ autologous ¹³⁷Cs-irradiated (2500 rads) non-T stimulator cells from a single donor. The medium employed consists of RPMI-1640 supplemented with 20 mM HEPES (Invitrogen), 5×10⁻⁵ M 2-mercaptoethanol (BDH, Montreal, QC), 4 mM L-glutamine (Invitrogen), 100 U/ml penicillin (Invitrogen), and 100 μg/ml streptomycin sulfate, with the addition of 10% fresh human serum autologous to the responder T-cell donor for the AMLR. Varying concentrations of purified recombinant human AFP, human serum albumin, anti-human AFP monoclonal antibody clone #164 (125 μg/ml final concentration in culture) (Leinco Technologies, St. Louis, Mo.) are added at the initiation of cultures. AMLR cultures are incubated for 4 to 7 days, at 37° C. in 95% air and 5% CO₂. At the indicated intervals, DNA synthesis is assayed by a 6 hour pulse with 1 μCi of ³H-thymidine (specific activity 56 to 80 Ci/mmole; ICN Radioisotopes, Cambridge, Mass.). The cultures are harvested on a multiple sample harvester (Skatron, Sterling, Va.), and the incorporation of ³H-TdR is measured in a Packard 2500 TR liquid scintillation counter. Results are expressed as mean cpm±the standard error of the mean of triplicate or quadruplicate cultures.

The immunosuppressive activity of a candidate AFP polypeptide or fragment, derivative, or analog within the scope of the present invention can be assessed by its ability to suppress human autologous mixed lymphocyte reactions (AMLR). Generally, the candidate AFP polypeptide, fragment, or derivative is tested for its ability to inhibit the proliferative response of autoreactive lymphocytes stimulated by autologous non-T-cells, by measuring lymphocyte autoproliferation throughout a time course of 4 to 7 days. Suppression of AMLR in a dose-dependent manner is demonstrated by results from dose-response studies performed at the peak of T-cell autoproliferation where an AFP polypeptide or fragment, derivative, or analog is added at the initiation of cultures. Furthermore, parallel viability studies can be used to establish that the inhibitory activity of an AFP polypeptide or fragment, derivative, or analog on human autoreactive T-cells is not due to non-specific cytotoxic effects.

A third assay of AFP polypeptide or fragment, derivative, or analog activity can be performed using a myelin oligodendrocyte glycoprotein (MOG) mouse model of experimental autoimmune encephalomyelitis (EAE). In this in vivo assay, genetically susceptible strains of mice are subcutaneously immunized with MOG emulsified in Complete Freund's Adjuvant (CFA), which leads to the development of EAE in the animals. A candidate AFP polypeptide or fragment, derivative, or analog is administered to a selected group of mice on a daily basis, beginning prior to, at the same time, or subsequent to the start of the administration of MOG to the animals. The symptoms of EAE in these animals are monitored and compared to those in a control group (e.g., those receiving only saline injections) over a certain time period, e.g., 30 days. Severity of EAE in each animal is given a score between 1-5 based on defined clinical symptoms; the average score of animals in a group indicates the disease state of the group. Biologically active AFP proteins or fragments will reduce the severity of EAE in animals receiving MOG compared to controls (e.g., at least a 50% reduction in the severity of disease after 30 days of treatment).

A fourth assay is available that relies on the ability of a candidate AFP within the scope of this invention to inhibit inflammatory cytokines induced by in vitro cultures of mitogen-stimulated splenocytes from naive mice (e.g., as described in Hooper and Evans, J. Reprod. Immunol. 16: 83-961,1989; and Kruisbeek, in Current Protocols in Immunology, Vol. 1, Section 3.1.1-3.1.5, 2000). Splenocytes are stimulated with phytohemagglutinin (PHA), conconalavin A (ConA), or lipopolysaccharide (LPS) in the presence of increasing concentrations of an AFP for 24 hours. Human serum albumin is used as a negative control for the assays. A 10 point dose response study has shown that biologically active AFP inhibits or substantially inhibits the secretion of PHA induced IFN-γ in a reproducible manner.

### Immunomodulatory Agents for Use in the Compositions and Methods of the Invention

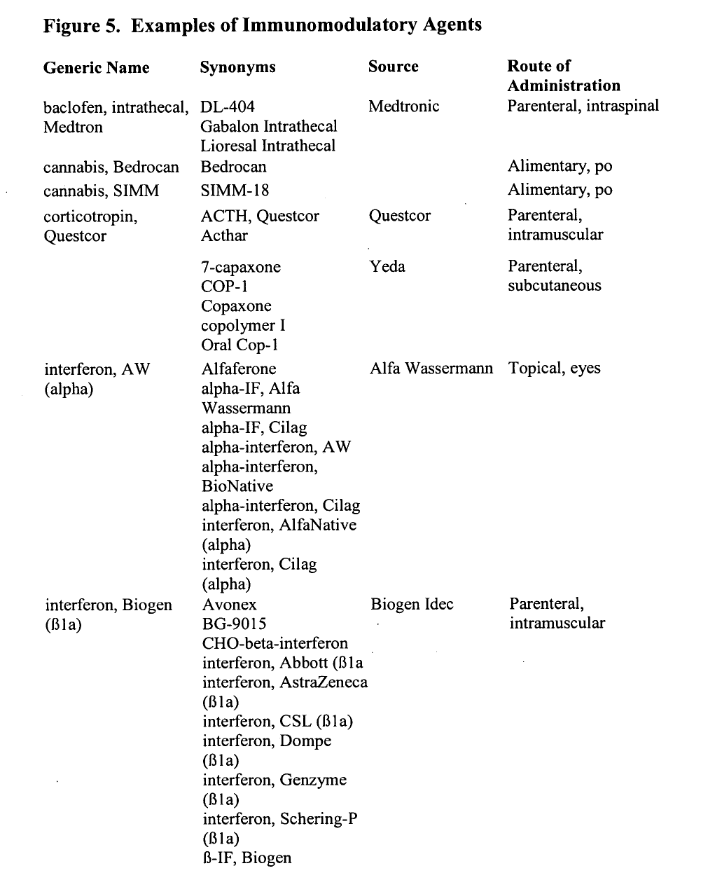

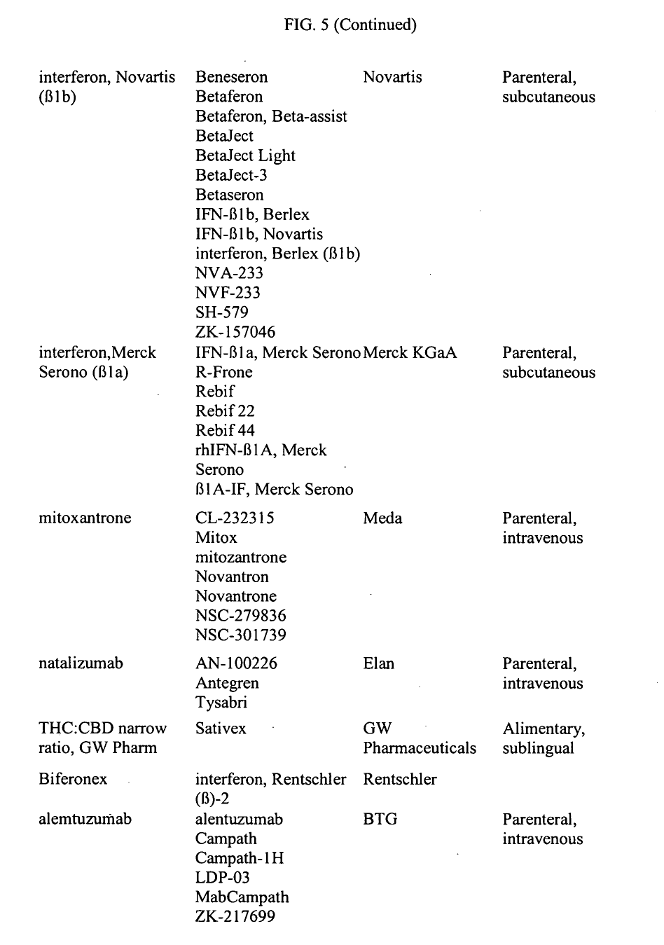

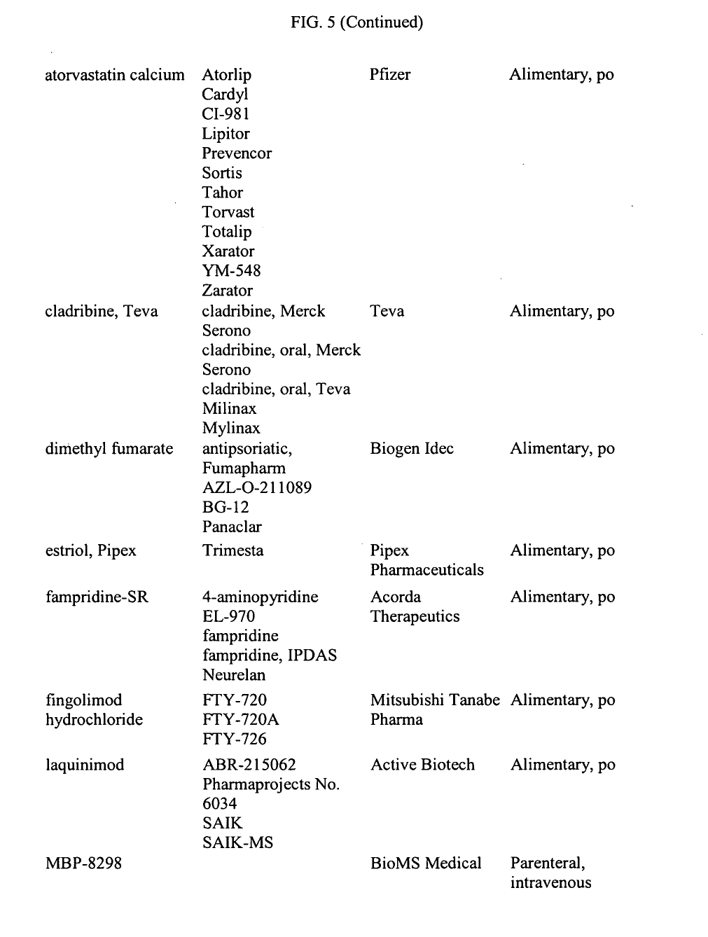

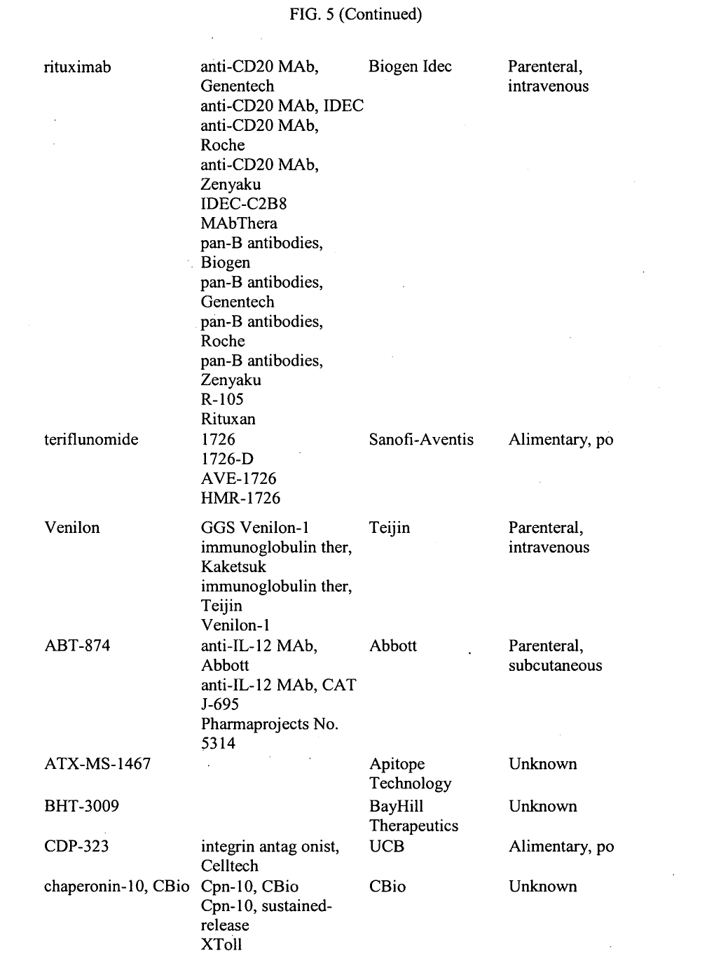

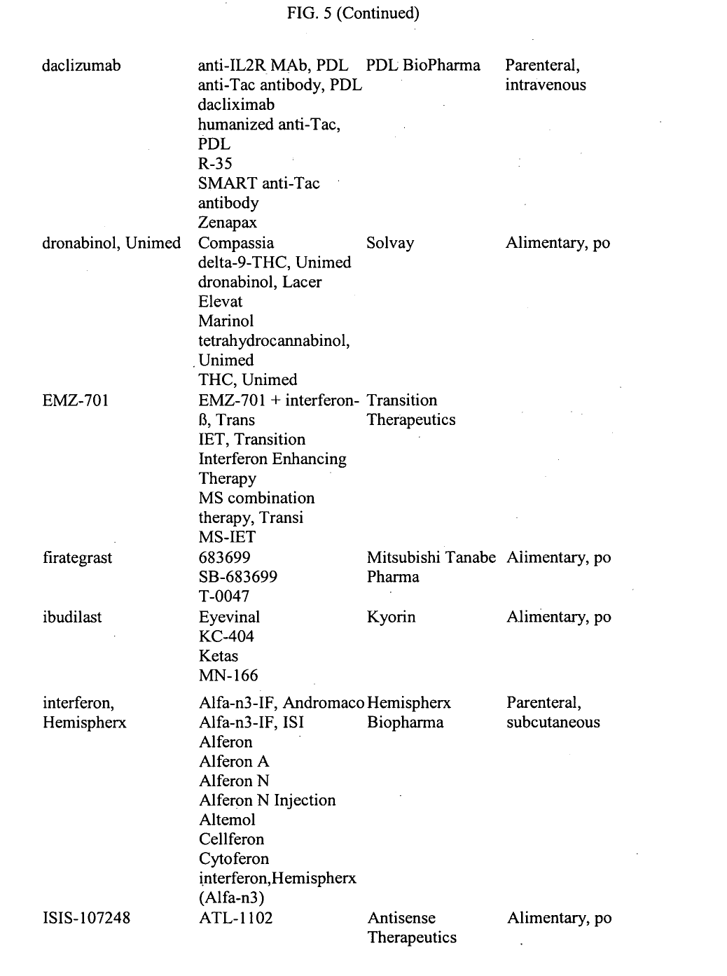

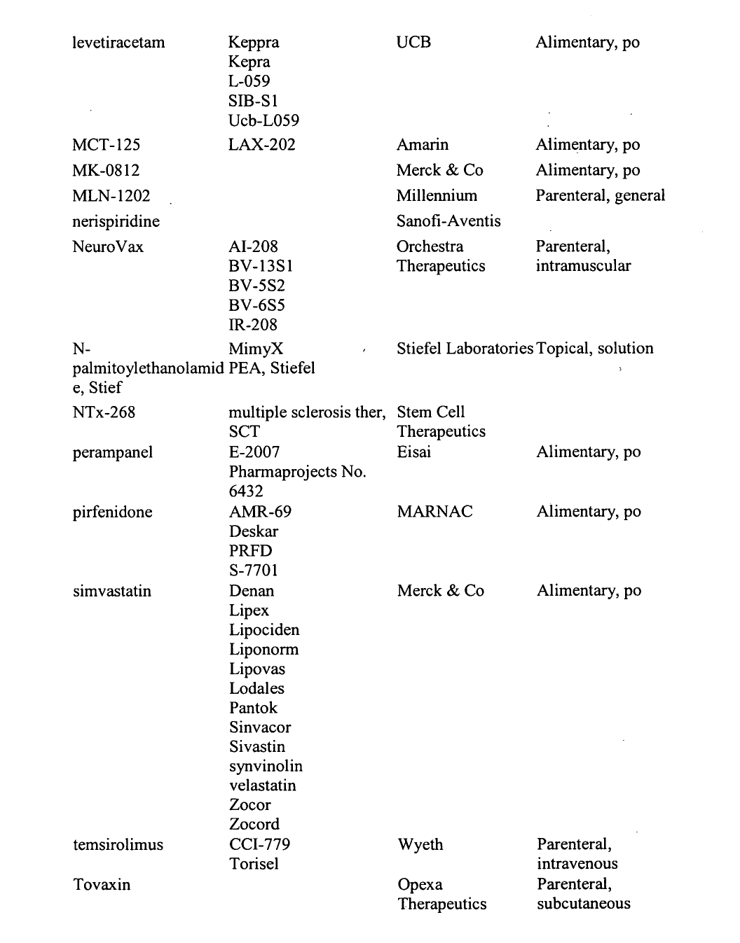

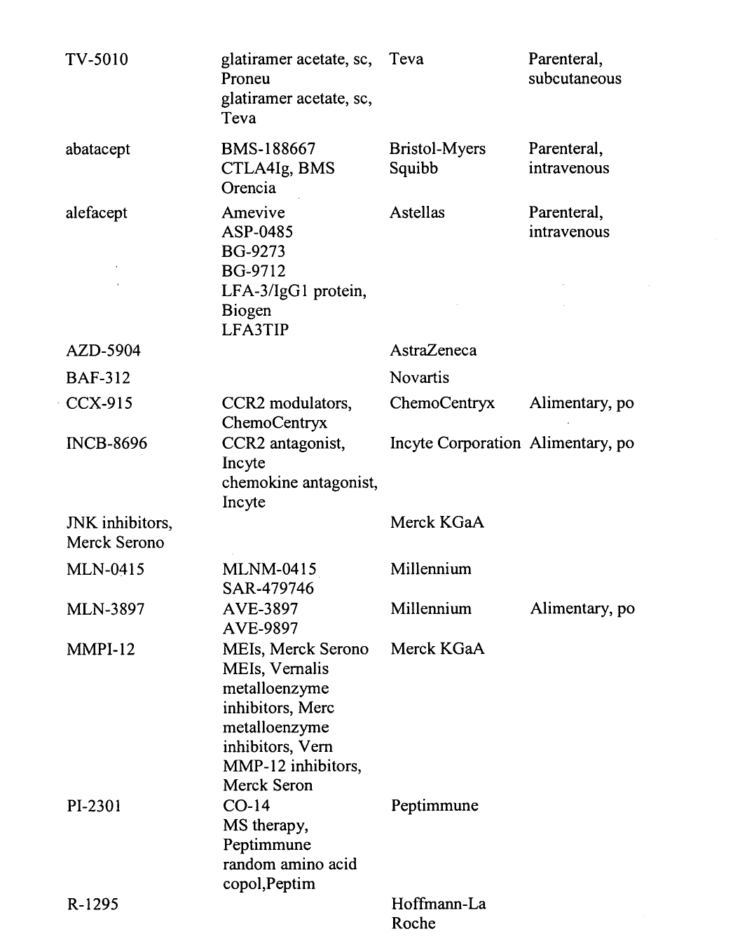

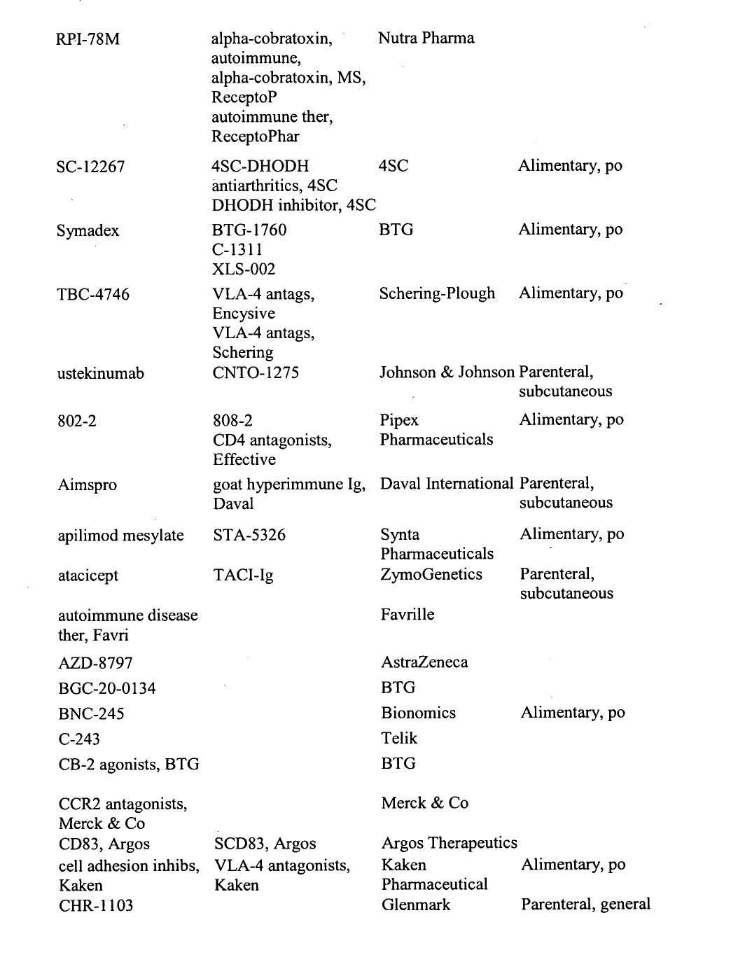

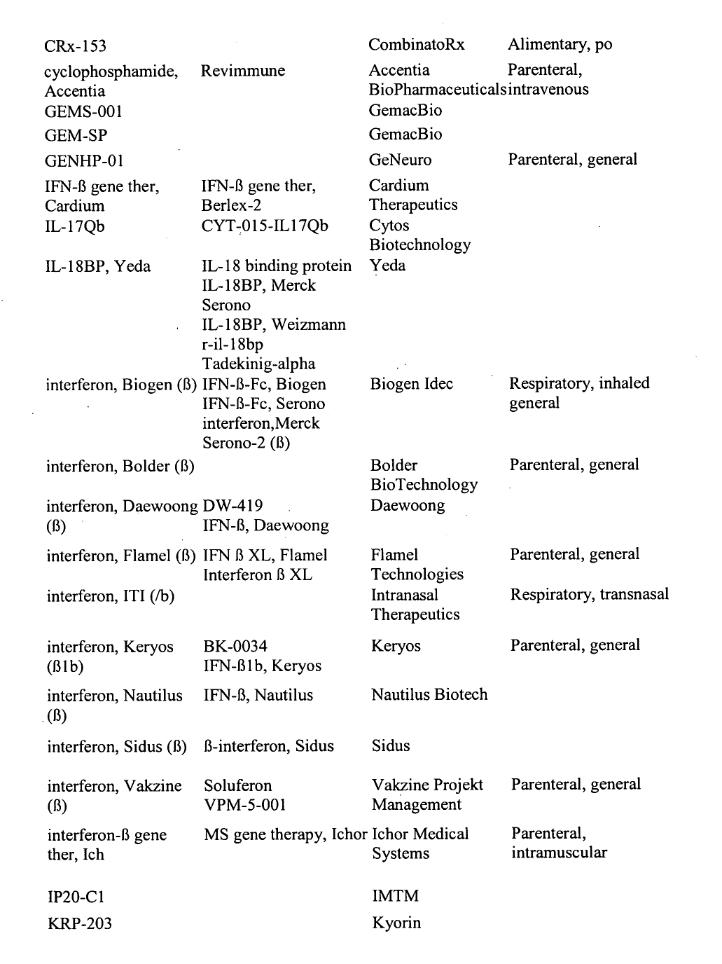

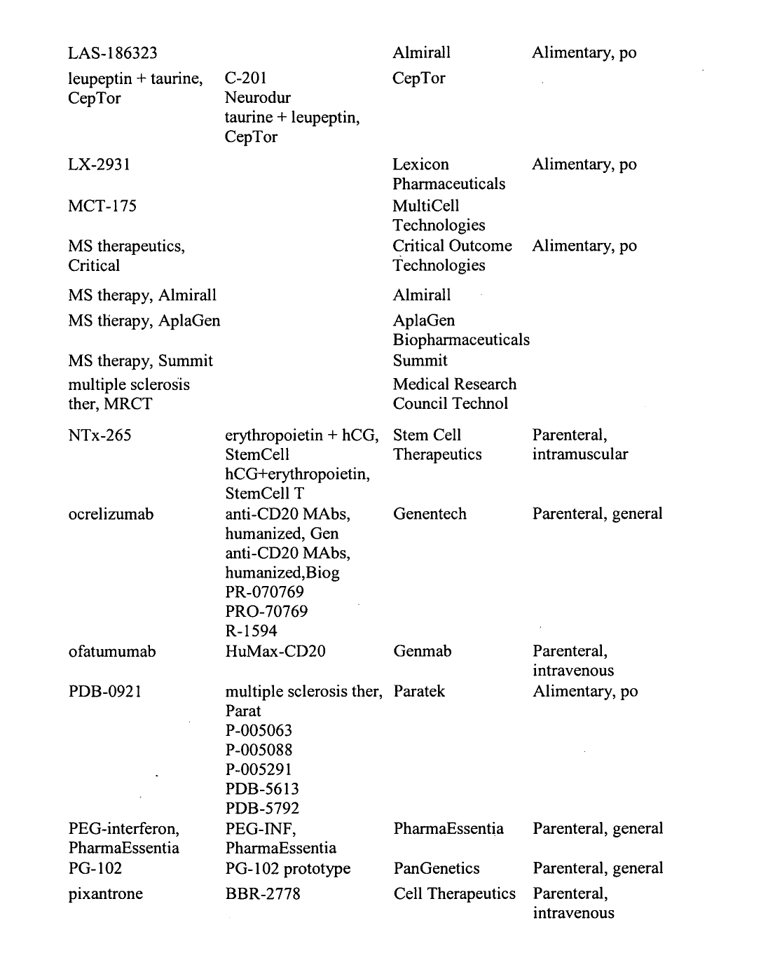

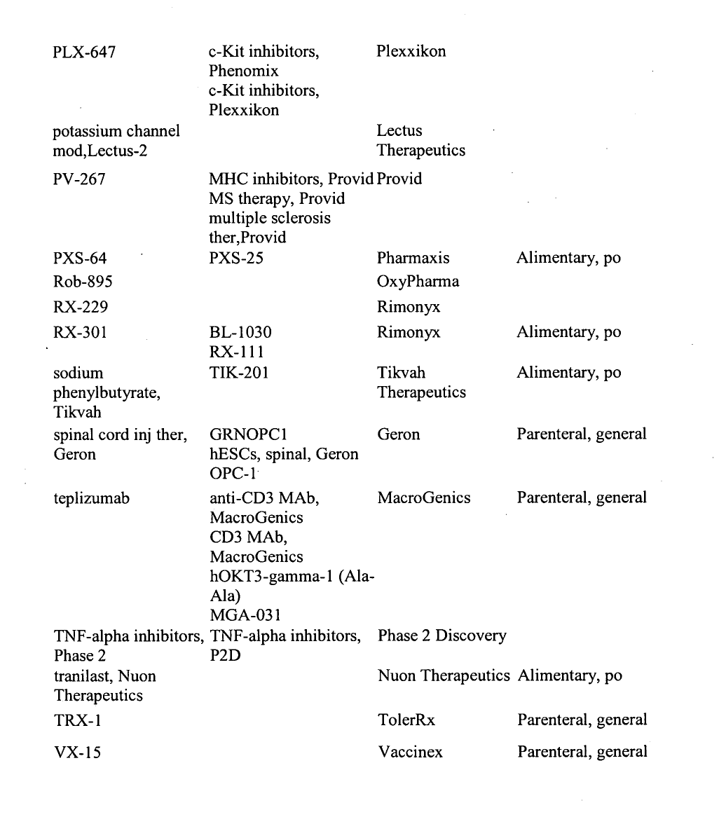

The compositions of the present invention also include one or more immunomodulatory agents for use in the treatment of MS. Immunomodulatory agents, as defined herein, are (1) proteins having an amino acid sequence substantially identical (e.g., at least 70%, 75%, 80%, 85%, 90%, 95%, 96%, 97%, 98%, 99%, or even 100% identical) to the sequence of a human IFN-α (e.g., IFN-α-1a, IFN-α-1b, IFN-α-2a, and IFN-α-2b; SEQ ID NOS: 11, 12, 13, and 14, respectively), a human IFN-β (e.g., IFN-β-1a and IFN-β-1b; SEQ ID NOS: 15 and 16, respectively), a human IFN-γ (e.g., SEQ ID NO: 17), or a human IFN-τ (SEQ ID NO: 18), or a peptide, such as glatiramer acetate (Copaxone®); (2) small molecules (e.g., BG12 (fumarate), fingolimod (FTY-720), laquinimod, teriflunomide, or atorvastatin, or a molecule that demonstrates the same or similar biological activity to an interferon (e.g., at least 50%, 60%, 70%, 75%, 80%, 85%, 90%, 95%, or 100% of the activity of a human IFN-α, a human IFN-β, a human IFN-γ, or a human IFN-τ in the ability to suppress EAE in a mouse model)); (3) antibodies (e.g., all. or part of a monoclonal antibody (e.g., an α4 integrin-binding antibody, such as nataluzimab; an IL-2 receptor-binding antibody, such as daclizumab; a CD20-binding antibody, such as rituximab; an IL-12 binding antibody, such as ABT-874; and a CD52-binding antibody, such as alemtuzumab), a polyclonal antibody, or an antibody fusion protein); (4) peptides (e.g., MBP-8289, NBI-5788, and T cell receptor peptide (Neurovax®)); or (5) DNA vaccines (e.g., BNT-3009-01). Additional examples of immunomodulatory agents are listed in FIG. 5.

Preferred immunomodulatory agents have the ability to decrease (e.g., by at least 10%, 15%, 20%, 25%, 30%,35%, 40%, 45%, 50%, 60%, 70%, 80%, 90%, or even 100%) the severity of one or more symptoms of MS, or the ability to prevent or decrease (e.g., by at least 10%, 15%, 20%, 25%, 30%, 35%, 40%, 45%, 50%, 60%, 70%, 80%, 90%, or even 100%) the progression of MS in a patient (e.g., the demyelination of nerves and the frequency or severity of one or more symptoms of MS) relative to those MS patients that do not receive the immunomodulatory agent or those patients who receive a placebo.

Non-limiting examples of immunomodulatory agents include those proteins that have at least 50% (more preferably at least 60%, 70%, 75%, 80%, 90%, 95% or 100%) amino acid sequence identity to a human IFN-α, -β, -γ, or -τ, and that have at least 50% (more preferably at least 50%, 60%, 70%, 75%, 80%, 85%, 90%, 95%, or 100%) of the activity of human IFN-β-1a in the ability to suppress EAE in a mouse model, which is similar to the assay system used for verifying AFP activity. Non-limiting examples of immunomodulatory agents include both naturally-purified interferons and recombinantly-produced interferons.

Recombinant production of interferons is routinely practiced in the field of biomedical research and for various clinical purposes. For instance, human IFN-β-1a, naturally a glycoprotein of 166 amino acids, has been produced in a Chinese hamster ovary cell line as Avonex® (Biogen, Cambridge, Mass.) or Rebif® (Serono, Geneva, Switzerland), which may present a glycosylation pattern different from that of its naturally occurring human counterpart.

Similarly, IFN-β-1b has been recombinantly produced in a strain of E. coli cells as Betaseron® (Berlex, Wayne, N.J.) or Betaferon (Schering AG, Berlin, Germany). Although naturally occurring human IFN-β-1b is also a glycosylated protein, the bacterially produced IFN-β-1b is not. Furthermore, in some cases, the cysteine residue at position 17 of human IFN-β-1b is substituted by a serine to prevent undesired disulfide bond formation. In other cases, the first amino acid of human IFN-β-1b, methionine, is deleted such that the recombinant protein has only 165 amino acids. Another known modified version of human IFN is Tauferon™ (Pepgen, Alameda, Calif.), which shares about 55% sequence homology to human IFN-α and is suitable for oral administration. This modified human IFN protein is described in, e.g., WO05/044297, which is incorporated herein by reference.

Non-limiting exemplary immunomodulatory agents which are recombinant interferons include, Roferon-A® (IFN-α-2a), Intron-A® (IFN-α-2b), Rebetron® (IFN-α-2b), Alferon-N® (IFN-α-n3), Peg-Intron® (IFN-α-2b covalently conjugated with monomethoxy polyethylene glycol), Infergen® (a non-naturally occurring type 1 interferon with 88% homology to IFN-α-2b), Actimmune® (IFN-γ-1b), and Pegasys® (pegylated IFN-α-1a).

Additional immunomodulatory agents of the invention include Copaxone®, Novantron®, laquinimod, teriflunomide, atorvastatin, natalizumab, daclizumab, rituximab, ABT-874, alemtuzumab, MBP-8289, NBI-5788, Neurovax®, and BNT-3009-01.

### Supplemental Therapeutic Agents for Use in the Combination Therapies of the Invention

In addition to an AFP (or a biologically active fragment, derivative, or analog thereof) and one or more immunomodulatory agents, the combination therapies of the invention may also, but need not, include the co-administration of one or more secondary agents, such as those listed below.

Disease-Modifying Anti-Rheumatic Drugs (DMARDs)

Several drugs are known in the field and presently used to treat patients with inflammatory disorders. If desired, the compositions of the invention may be administered in conjunction with one or more DMARDs. Non-limiting examples of DMARDs that may be used in the present invention include, but are not limited to auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

Methotrexate is an example of a DMARD that can be used in one embodiment of the combination treatment method of this invention. Methotrexate, also known as Amethopterin, RHEUMATREX® (Lederle Pharmaceutical), or FOLEX® (Aventis), is an antimetabolite that competitively and reversibly inhibits dihydrofolate reductase (DHFR), an enzyme that is part of the folate synthesis metabolic pathway.

The chemical name for methotrexate is N-[4-[[(2,4-diamino-6-pteridinyl)methyl]methylamino]benzoyl]-L-glutamic acid, although it is commonly present in the form of a sodium salt in pharmaceutical compositions and its amount in such compositions is determined by equivalence to the free acid. Therefore, when a composition is said to contain 10 mg of methotrexate, a greater weight of a sodium salt of methotrexate may be present in the composition. Methotrexate is a generic drug that has been in use for many years and is commercially available through various suppliers. For instance, methotrexate is manufactured and marketed by both Pfizer and Wyeth.

Non-Steroidal Anti-Inflammatory Drugs (NSAIDs)

If desired, the compositions of the invention may be administered in conjunction with one or more NSAIDs. Non-limiting examples of NSAIDs that may be used in the present invention include naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, tolmetin, and COX-2 inhibitors such as rofecoxib, celecoxib, valdecoxib, or lumiracoxib.

Corticosteroids

If desired, the compositions of the invention may be administered in conjunction with one or more corticosteroids. Corticosteroids are naturally occurring or synthetic compounds characterized by a hydrogenated cyclopentanoperhydrophenanthrene ring system. Naturally occurring corticosteroids are generally produced by the adrenal cortex. Synthetic corticosteroids may be halogenated. Exemplary corticosteroids which may be used in the present invention include algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha-9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Particularly desirable corticosteroids are prednisolone, cortisone, dexamethasone, hydrocortisone, methylprednisolone, fluticasone, prednisone, triamcinolone, and diflorasone.

### Pharmaceutical Compositions

Pharmaceutical compositions of the invention contain a therapeutically effective amount of AFP (or a biologically functional fragment, derivative, or analog thereof) and/or one or more immunomodulatory agents. The active ingredients, an AFP and one or more immunomodulatory agents, may be administered in the same pharmaceutical composition, or they may be present in two separate pharmaceutical compositions, both of which are administered to the patient (e.g., coextensively or non-coextensively). The compositions can be formulated for use in a variety of drug delivery systems. One or more physiologically acceptable excipients or carriers can also be included in the compositions for proper formulation. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions are intended for parenteral, intranasal, topical, oral, or local administration, such as by a transdermal means, for prophylactic and/or therapeutic treatment. Commonly, the pharmaceutical compositions are administered parenterally (e.g., by intravenous, intramuscular, or subcutaneous injection), or by oral ingestion, or by topical application at areas affected by MS. Thus, the invention provides compositions for parenteral administration that comprise an AFP (or biologically active fragment, derivative, or analog thereof) and one or more immunomodulatory agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like. In different embodiments of the invention, the AFP and the one or more immunomodulatory agents may be formulated in the same or separate compositions for administration via the same or two different routes of administration.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of AFP (or a biologically active fragment, derivative, or fragment thereof) and one or more immunomodulatory agents, such as in a sealed package of tablets or capsules. The composition in solid form can also be packaged in a container for a flexible quantity, such as in a squeezable tube designed for a topically applicable cream or ointment.

The compositions containing an effective amount of an AFP (or biologically active fragment, derivative, or analog thereof) and one or more immunomodulatory agents can be administered for prophylactic and/or therapeutic treatments. In prophylactic applications, compositions of the invention containing an AFP (or biologically active fragment, derivative, or analog thereof) and/or one or more immunomodulatory agents are administered to a patient susceptible to or otherwise at risk of developing MS. Such an amount is defined to be a “prophylactically effective dose.” In this use, the precise amounts again depend on the patient's state of health, but generally range from about 0.5 mg to about 400 mg of AFP (or a biologically active fragment, derivative, or analog thereof) per dose (e.g., 10 mg, 50 mg, 100 mg, 200 mg, 300 mg, or 400 mg per dose) and from about 0.1 μg to about 300 mg of one or more immunomodulatory agents per dose (e.g., 10 μg, 30 μg, 50 μg, 0.1 mg, 10 mg, 50 mg, 100 mg, or 200 mg per dose). A dose of the AFP and/or immunomodulatory agent can be administered prophylactically to a patient one or more times per hour, day, week, month, or year (e.g., 2, 4, 5, 6, 7, 8, 9, 10, 11, or 12 times per hour, day, week, month, or year). More commonly, a single dose per week of an AFP and/or an immunomodulatory agent is administered to a patient.

In therapeutic applications, compositions are administered to a patient (e.g., a human patient) already suffering from MS in an amount sufficient to cure or at least partially arrest or alleviate one or more of the symptoms of the disease and its complications. An amount adequate to accomplish this purpose is defined as a “therapeutically effective dose.” Amounts effective for this use may depend on the severity of the disease or condition and general state of the patient, but generally range from about 0.5 mg to about 400 mg of AFP (or biologically active fragment, derivative, or analog thereof) per dose (e.g., 10 mg, 50 mg, 100 mg, 200 mg, 300 mg, or 400 mg per dose) and from about 0.1 μg to about 1.2 g of one or more immunomodulatory agents per dose (e.g., 10 μg, 30 μg, 50 μg, 0.1 mg, 10 mg, 50 mg, 100 mg, 200 mg, 300 mg, 500 mg, 700 mg, or 1.0 g per dose). A dose of the AFP and/or immunomodulatory agent can be administered therapeutically to a patient one or more times per hour, day, week, month, or year (e.g., 2, 4, 5, 6, 7, 8, 9, 10, 11, or 12 times per hour, day, week, month, or year). More commonly, a single dose per week of an AFP and/or an immunomodulatory agent is administered to a patient.

In several embodiments, the patient may receive an AFP (or biologically active fragment, derivative, or analog thereof) in the range of about 0.5 to about 400 mg per dose one or more times per week (e.g., 2, 3, 4, 5, 6, or 7 or more per week), preferably about 5 mg to about 300 mg per dose one or more times per week, and even more preferably about 5 mg to about 200 mg per dose one or more times per week. The patient may also receive a biweekly dose of an AFP (or biologically active fragment, derivative, or analog thereof) in the range of about 50 mg to about 800 mg or a monthly dose of an AFP (or biologically active fragment, derivative, or analog thereof) in the range of about 50 mg to about 1,200 mg.

In other embodiments, an AFP may be administered to a patient in a typical dosage range of about 0.5 mg per week to about 400 mg per week, about 1.0 mg per week to about 300 mg per week, about 5 mg per week to about 200 mg per week, about 10 mg per week to about I 00 mg per week, about 20 mg per week to about 80 mg per week, about 100 mg per week to about 300 mg per week, or about 100 mg per week to about 200 mg per week. An AFP may be administered in the range of about 0.5 mg every other day to about 100 mg every other day, preferably about 5 mg every other day to about 75 mg every other day, more preferably about 10 mg every other day to about 50 mg every other day, and even more preferably 20 mg every other day to about 40 mg every other day. An AFP may also be administered in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In several embodiments, the patient may receive an immunomodulatory agent in the range of about 30 μg to about 300 mg per dose one or more times per week (e.g., 2, 3, 4, 5, 6, or 7 or more times a week), preferably about 30 μg to about 200 mg per dose one or more times per week, and more preferably about 30 μg to about 100 mg per dose one or more times per week. The patient may also receive a biweekly, triweekly, or montly dose of an immunomodulatory agent in the range of about 30 μg to about 1.2 g, preferably a dose in the range of about 50 μg to about 1,000 mg, more preferably a dose in the range of about 100 μg to about 500 mg.

In some embodiments where the immunomodulatory agent administered is Avonex®, the patient receives a typical dosage in.the range of about 15 μg per week to about 75 μg per week, preferably about 20 μg per week to about 50 μg per week, more preferably about 25 μg per week to about 40 μg per week, and even more preferably about 30 μg per week to 40 μg per week. The patient may also receive an AFP polypeptide (or biologically active fragment ther&of) in the range of about 0.5 mg per week to about 100 mg per week, preferably about 5 mg per week to about 75 mg per week, more preferably about 10 mg per week to about 50 mg per week, and even more preferably about 20 mg per week to about 40 mg per week.

In another exemplary embodiment where the immunomodulatory agent administered is Betaseron®, the typical dosage administered may be in the range of about 6 μg every other day to about 2.0 mg every other day, preferably about 50 μg every other day to about 1.0 mg every other day, more preferably about 100 μg every other day to about 500 μg every other day, and even more preferably about 250 μg every other day to about 500 μg every other day. The patient may also receive an AFP polypeptide (or biologically active fragment thereof) in the range of about 0.5 mg every other day to about 100 mg every other day, preferably about 5 mg every other day to about 75 mg every other day, more preferably about 10 every other day to about 50 mg every other day, and even more preferably about 20 mg every other day to about 40 mg every other day.

In another embodiment, where the immunomodulatory agent is Rebif®, the typical dosage administered may be in the range of about 4.4 μg three times per week to about 100 μg three times per week, preferably about 10 μg three times per week to about 75 μg three times per week, more preferably about 15 μg three times per week to about 50 μg three times per week, and even more preferably about 22 μg three times per week to about 44 μg three times per week. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Tauferon™, the typical dosage administered may be in the range of about 0.1 mg a day to about 40 mg a day, preferably about 0.1 mg a day to about 10 mg a day, more preferably about 1 mg a day to about 10 mg a day, and even more preferably about 2 mg a day to about 5 mg a day. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Roferon-A®, the typical dosage administered may be in the range of about 2.0×10⁶ IU a day to about 36.0×10⁶ IU a day, preferably about 3.0×10⁶ IU a day to about 36.0×10⁶ IU a day, more preferably about 5.0×10⁶ IU a day to about 30.0×10⁶ IU a day, and even more preferably about 5.0×10⁶ IU a day to about 25.0×10⁶ IU a day. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Intron-A®, the typical dosage administered may be in the range of about 5.0×10⁶ IU a week to about 35.0×10⁶ IU a week, preferably about 6.0×10⁶ IU a week to about 35.0×10⁶ IU a week, more preferably about 6.0×10⁶ IU a week to about 30.0×10⁶ IU a week, and even more preferably about 25.0×10⁶ IU a week to about 35.0×10⁶ IU a week. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Rebetron®, the typical dosage administered may be in the range of about 0.5×10⁶ IU a week to about 10.0×10⁶ IU a week, preferably about 1.0×10⁶ IU a week to about 10.0×10⁶ IU a week, more preferably about 2.0×10⁶ IU a week to about 10.0×10⁶ IU a week, and even more preferably about 5.0×10⁶ IU a week to about 10.0×10⁶ IU a week. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In some embodiments where the immunomodulatory agent administered is Peg-Intron®, the patient receives a typical dosage in the range of about 15 μg per week to about 150 μg per week, preferably about 20 μg per week to about 150 μg per week, more preferably about 50 μg per week to about 150 μg per week, and even more preferably about 50 μg per week to 100 μg per week. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Alferon-N®, the typical dosage administered may be in the range of about 0.05×10⁶ IU a day to about 15.0×10⁶ IU a day, preferably about 0.1×10⁶ IU a day to about 15.0×10⁶ IU a day, more preferably about 1.0×10⁶ IU a day to about 15.0×10⁶ IU a day, and even more preferably about 2.0×10⁶ IU a day to about 15.0×10⁶ IU a day. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In some embodiments where the immunomodulatory agent administered is Infergen®, the patient receives a typical dosage in the range of about 2 μg per day to about 30 μg per day, preferably about 5 μg per day to about 30 μg per day, more preferably about 5 μg per day to about 25 μg per day, and even more preferably about 5 μg per day to 20 μg per day. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Actimmune®, the typical dosage administered may be in the range of about 0.5×10⁶ IU a week to about 30.0×10⁶ IU a week, preferably about 1.0×10⁶ IU a week to about 30.0×10⁶ IU a week, more preferably about 5.0×10⁶ IU a week to about 30.0×10⁶ IU a week, and even more preferably about 5.0×10⁶ IU a week to about 10.0×10⁶ IU a week. Actimmunee may also be administered in the range of about 40 μg a week to about 600 μg a week, preferably about 100 μg a week to about 600 μg a week, more preferably about 150 μg a week to about 600 μg a week, and even more preferably about 200 μg a week to about 600 μg a week. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Pegasys®, the typical dosage administered may be in the range of about 10 μg a week to about 300 μg a week, preferably about 50 μg a week to about 300 μg a week, more preferably about 50 μg a week to about 200 μg a week, and even more preferably about 100 μg a week to about 200 μg a week. The patient may also receive an AFP polypeptide (or biologically active fragment thereof) in the range of about 0.5 mg three times per week to about 100 mg three time per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Novantrone®, the typical dosage administered may be in the range of about 0.2 mg/m² a week to about 80 mg/m² a week, preferably about 1.0 mg/m² a week to about 80 mg/m² a week, more preferably about 5.0 mg/m² a week to about 80 mg/m² a week, and even more preferably about 20.0 mg/m² a week to about 80 mg/m² a week. The patient may also receive an AFP polypeptide (or biologically active fragment thereof) in the range of about 0.5 mg three times per week to about 100 mg three time per week, preferably about 5 mg three times per week to about 75 mg three times per week, more preferably about 10 mg three times per week to about 50 mg three times per week, and even more preferably about 20 mg three times per week to about 40 mg three times per week.

In another embodiment, where the immunomodulatory agent is Copaxone®, the typical dosage administered may be in the range of about 0.1 mg a day to about 40 mg a day, preferably about 1.0 mg a day to about 40 mg a day, more preferably about 5.0 mg a day to about 40 mg a day, and even more preferably about 10.0 mg a day to about 40 mg a day. The patient may also receive an AFP polypeptide (or biologically active fragment thereof) in the range of about 0.5 mg three times per week to about 100 mg three times per week, preferably about 5 mg three times per week to about 75 mg three times per week, more preferably about 10 mg three times per week to about 50 mg three times per week, and even more preferably about 20 mg three times per week to about 40 mg three times per week.

In non-limiting embodiments of the methods of the present invention, an AFP (or biologically active fragment, derivative, or analog thereof) and one or more immunomodulatory agents are administered to a patient: continuously for 1, 2, 3, or 4 hours; 1, 2, 3, or 4 times a day; every other day or every third, fourth, fifth, or sixth day; 1, 2, 3, 4, 5, 6, 7, 8, 9, or 10 times a week; biweekly; 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 times a month; bimonthly; 1, 2, 3, 4, 5, 6, 7, 8, 9, or 10 times every six months; 1, 2, 3, 4, 5, 6, 7, 8, 9, 10, 11, 12, 13, 14, 15, 16, 17, 18, 19, or 20 times a year; or biannually. The AFP (and biologically active fragment, derivative, or analog thereof) and one or more immunomodulatory agents may be administered at different frequencies during a therapeutic regime (i.e., administered at a higher frequency in the later stages of MS (e.g., administered once a week in the initial stages of MS and administered three times a week a later stage of MS) or administered at a higher frequency in the early stages of MS (e.g., administered three times a week during the initial stages of MS and administered once a week at a later stage of MS)). In additional embodiments, the AFP (or biologically active fragment, derivative, or analog thereof) and the one or more immunomodulatory agents may be administered to a patient at the same frequency or at a different frequency.

The amount of one or more immunomodulatory agents and AFP polypeptide (or biologically active fragment, derivative, or analog thereof) required to achieve the desired therapeutic effect depends on a number of factors, such as the specific immunomodulatory agent(s) chosen, the mode of administration, and clinical condition of the recipient. A skilled artisan will be able to determine the appropriate dosages of one or more immunomodulatory agents and AFP (or biologically active fragment, derivative, or analog thereof) to achieve the desired results.

The coadministration of AFP (or a biologically active fragment, derivative, or analog thereof) and one or more immunomodulatory agents according to the method of this invention refers to the use of the two active ingredients in the same general time period or using the same general administration method. It is not always necessary, however, to administer both at the exact same time. For instance, if an AFP and one or more immunomodulatory agents are administered to a patient suffering from MS in two separate pharmaceutical compositions, the two compositions need not be delivered to the patient during the same time period or even during two partially overlapping time periods. In some cases, the administration of the second agent (e.g., an AFP) may begin shortly after the completion of the administration period for the first agent (e.g., IFN-β-1a), or vice versa. The time gap between the two administration periods may vary from one day to one week or one month. In some cases, one therapeutic agent (e.g., an AFP) may be administered first with the second agent (e.g., an IFN) in a separate time period, and subsequently administered without the second in a following period. A typical schedule of this type may require a higher dosage of the first therapeutic agent in the first co-administration period and a lower dosage in the second period and vice versa. The same applies for the second agent.

Single or multiple administrations of the compositions comprising an effective amount of AFP (or biologically active fragment, derivative, or analog thereof) and/or one or more immunomodulatory agents can be carried out with dose levels and pattern being selected by the treating physician. The dose and administration schedule can be determined and adjusted based on the severity of multiple sclerosis in a patient, which may be monitored throughout the course of treatment according to the methods commonly practiced by clinicians or those described herein.

Dosages for Secondary Agents

In addition to an AFP (or a biologically active fragment, derivative, or analog thereof) and one or more immunomodulatory agents, the combination therapies of the invention may also, but need not, include the co-administration of one or more secondary agents (e.g., a DMARD, NSAID, or corticosteroid). Dosages for these secondary agents are described below.

As a secondary agent, a DMARD can be administered to a patient in the range of about 0.1 mg to 3,000 mg per dose one or more times per week (e.g., 2, 3, 4, 5, 6, or 7 or more times per week), 0.1 mg to 2,500 mg per dose one or more times per week, 0.1 mg to 2,000 mg per dose one or more times per week, 0.1 mg to 1,500 mg per dose one or more times per week, 0.1 mg to 1,000 mg per dose one or more times per week, 0.1 mg to 800 mg per dose one or more times per week, 0.1 mg to 600 mg per dose one or more times per week, 0.1 mg to 500 mg per dose one or more times per week, 0.1 mg to 400 mg per dose one or more times per week, 0.1 mg to 300 mg per dose one or more times per week, 0.1 mg to 250 mg per dose one or more times per week, 0.1 mg to 200 mg per dose one or more times per week, 0.1 mg to 150 mg per dose one or more times per week, 0.1 mg to 100 mg per dose one or more times per week, or 0.1 mg to 50 mg per dose one or more times per week.

As a secondary agent, an NSAID can be administered to a patient in the range of 0.1 mg to 1,500 mg per dose one or more times per week (e.g., 2, 3, 4, 5, 6, or 7 or more times per week), 0.1 mg to 1,200 mg per dose one or more times per week, 0.1 mg to 1,000 mg per dose one or more times per week, 0.1 mg to 800 mg per dose one or more times per week, 0.1 mg to 600 mg per dose one or more times per week, 0.1 mg to 500 mg per dose one or more times per week, 0.1 mg to 400 mg per dose one or more times per week, 0.1 mg to 300 mg per dose one or more times per week, 0.1 mg to 200 mg per dose one or more times per week, 0.1 mg to 150 mg per dose one or more times per week, 0.1 mg to 100 mg per dose one or more times per week, 0.1 mg to 80 mg per dose one or more times per week, 0.1 mg to 60 mg per dose one or more times per week, 0.1 mg to 40 mg per dose one or more times per week, 0.1 mg to 20 mg per dose one or more times per week, or 0.1 mg to 10 mg per dose one or more times per week.

As a secondary agent, a corticosteroid can be administered to a patient in the range of 0.1 mg to 1,500 mg per dose one or more times per week (e.g., 2, 3, 4, 5, 6, or 7 or more times per week), 0.1 mg to 1,200 mg per dose one or more times per week, 0.1 mg to 1,000 mg per dose one or more times per week, 0.1 mg to 800 mg per dose one or more times per week, 0.1 mg to 600 mg per dose one or more times per week, 0.1 mg to 500 mg per dose one or more times per week, 0.1 mg to 400 mg per dose one or more times per week, 0.1 mg to 300 mg per dose one or more times per week, 0.1 mg to 200 mg per dose one or more times per week, 0.1 mg to 150 mg per dose one or more times per week, 0.1 mg to 100 mg per dose one or more times per week, 0.1 mg to 80 mg per dose one or more times per week, 0.1 mg to 60 mg per dose one or more times per week, 0.1 mg to 40 mg per dose one or more times per week, 0.1 mg to 20 mg per dose one or more times per week, or 0.1 mg to 10 mg per dose one or more times per week.

### Kits

The invention also provides kits for treating MS. The kits typically include a pharmaceutical composition containing an AFP polypeptide (or a biologically active fragment, derivative, or analog thereof) as well as a pharmaceutical composition containing one or more immunomodulatory agents, each in a therapeutically effective amount for treating MS. In the alternative, effective amounts of an AFP (or biologically active fragment, derivative, or analog thereof) and one or more immunomodulatory agents can be present in a single pharmaceutical composition. Optionally, the pharmaceutical composition(s) may contain one or more pharmaceutically acceptable excipients or may contain one or more secondary agents (e.g., a DMARD, corticosteroid, or NSAID).

Preferably, the kits include multiple packages of the single-dose pharmaceutical composition(s) containing an effective amount of an AFP (or biologically active fragment, derivative, or analog thereof) and/or one or more immunomodulatory agents. Optionally, instruments or devices necessary for administering the pharmaceutical composition(s) may be included in the kits. For instance, a kit of this invention may provide one or more prefilled syringes containing an effective amount of an AFP (or biologically active fragment, derivative, or analog thereof) and one or more prefilled syringes or tablets containing an effective amount of one or more immunomodulatory agents. Furthermore, the kits may also include additional components such as instructions or administration schedules for a patient suffering from MS to use the pharmaceutical composition(s) containing an AFP (or biologically active fragment, derivative, or analog thereof) and/or one or more immunomodulatory agents. Different embodiments of the kits of the invention may also contain one or more secondary agents (e.g., NSAID, DMARD, or corticosteroid).

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### Examples

The following examples are provided by way of illustration only and not by way of limitation. Those of skill in the art will readily recognize a variety of non-critical parameters that could be changed or modified to yield essentially the same or similar results.

### Example 1

### Functional Test of a Recombinant AFP Using MOGEAE Mouse Model

Efficacy experiments of a recombinant version of human AFP (rhAFP produced according to U.S. patent application publication No. 20040098755) were performed in a mouse model in which experimental autoimmune encephalomyelitis (EAE) is induced by immunization of genetically susceptible strains of mice with myelin antigen or peptides (myelin oligodendrocyte protein [MOG] or proteolipid protein [PLP]). This assay system is useful for determining the functionality of an AFP polypeptide or a biologically active AFP fragment of this invention.

Purpose of Study: The purpose of these studies was to perform with test compounds intended as therapeutics for MS, an autoimmune disease directly associated with the major histocompatibility complex (MHC) class II molecule HLA-DR2. The mouse experimental autoimmune encephalomyelitis (EAE) model was chosen for its relevance to human MS.

EAE Model Description and Features: Experimental Allergic Encephalomyelitis (EAE) is a demyelinating disease of the central nervous system. It serves as the animal model for Multiple Sclerosis (MS) (Govermanr, Lab. Anim. Sci. 46:482, 1996; Paterson, Clin. Immunol. Rev. 1:581, 1981). EAE can assume an acute, chronic, or relapsing-remitting disease course that is dependent upon the method of induction and type of animal used. Disease induction results in escalating degrees of ascending animal paralysis. The resulting paralysis is debilitating, but not painful, and most animals will show some degree of recovery even from advanced stages of EAE. Paralysis usually begins with a weakened tail, gradually followed by hind limb weakness progressing to paralysis, and less frequently front limb paralysis. EAE disease progression can be monitored with a scoring system that starts with the normal condition and ends when the mice become moribund. Since the severity of the disease varies from animal to animal there is no way to reliably predict whether an animal will recover. As a result, close monitoring is needed in this animal model.

EAE can be induced with components of the central nervous system (Levine and Sowinski, J. Immunol. 110:139, 1973; Fritz et al., J. Immunol. 130:1024, 1983) or peptides (Tuohy et al., J. Immunol. 140:1868, 1988; McFarlin et al., Science 179:478, 1973; and Linington et al., Eur. J. Immunol. 23:1364, 1993) and also via T cell transfer from an EAE-induced animal to normal recipient (Yamamura et al., J. Neurol. Sci. 76:269, 1986). Complete Freund's adjuvant (CFA) must be used with the proteins or peptides to effectively trigger the autoimmune response. CFA is often used in combination with pertussis toxin (Lee, Proc. Soc. Exp. Biol. Med. 89:263, 1955; Kamradt et al., J. Immunol. 147:3296, 1991) to increase the efficiency of immunization. It is not possible to administer analgesics to lessen any pain that may be associated with the CFA injections, as most analgesics affect the immune response that is an essential component of the model (Billiau, J. Leukoc. Biol. 70:849, 2001; Naiki et al., Int. J. Immunopharmacol. 13:235, 1991).

### Experimental Design and Methods

Induction of experimental MS-like disease syndrome: 50 Female mice (C57BL6) between 6 and 8 weeks of age, were immunized subcutaneously on day 0 (left paralumbar region) and day 7 (right paralumbar region) with an emulsion (125 μg per mouse) of myelin oligodendrocyte glycoprotein (mMOG-35-55 peptide) in CFA containing heat-killed Mycobacterium tuberculosis H37RA. In addition, mice were given pertussis toxin (Ptx) intraperitonealy, on days 0 and 2 post-immunization.

Disease monitoring: The initial signs of disease (weakened tail or paralysis) were observed beginning ˜10 days after the first immunization. Actively immunized mice were assessed daily through day 30 for clinical signs of EAE according to an established scale:

| | |
|---|---|
| 0 | No disease |
| 1 | Tail weakness |
| 2 | One or two weak hind limbs, sufficient to impair righting or 1 limb |
| | paralysis |
| 3 | paraplegic |
| 4 | quadraplegic |
| 5 | Moribund or dead |

The 50 mice were randomized into 5 groups of 10 mice each. One group of 10 animals received a saline injection to serve as an untreated EAE disease control. Four compounds were evaluated in the remaining 4 groups.

Mice were injected with 100 μl of test rhAFP or control material IP daily. These compounds are: 1-500 μg rhAFP; 1-500 μg Human Serum Albumin (control). Furthermore, depleting antibodies to specific leukocyte subsets (e.g., CD4⁺ cells) are employed as additional control(s) in some studies.

Mice were used in this study to assess the effect of rhAFP on disease progression in an experimental model of MS, EAE. Without treatment it was expected that many of the animals would develop signs and symptoms of EAE, namely progressive encephalopathy and paralysis.

In addition to daily monitoring of the animals for disease progression over a 30-day time course, animals were sacrificed at the end of the study and central nervous system tissues (brain and spinal cord) were harvested for immunohistochemical analysis of infiltrating, disease causing cells (i.e., CD4⁺ T cells).

Additionally, six to ten-day short-term studies were employed to assess the effect(s) of rhAFP administration on the induction phase of disease. In these shorter studies, draining lymph node cells were harvested for FACs analysis of immunologic cell subsets including but not limited to: T cells, CD4⁺]cells, regulatory T cells, and their activation markers. A fraction of harvested cells from each treatment group were assessed for in vitro proliferative response to a panel of stimuli to assess Ag-specific recall response to the immunizing antigen (Ag), MOG35-55 and Ag-nonspecific responses to a panel of mitogens (Concanavalin A, PHA, LPS). Supernatants from cultures set-up in the same fashion are analyzed for cytokines (IL-2, IL-4, IFN-γ, etc.).

### Example 2

### Synergistic Effect of AFP and Interferon β1a in MOG-EAE Mouse Model

The synergistic effect of recombinant human AFP and interferon β1a for treating EAE is tested in a study utilizing the MOG-EAE or PLP-EAE mouse model for MS.

The general experimental design is identical to Example 1. Briefly, 70 Female mice (C57BL6) between 6 and 8 weeks of age are immunized subcutaneously on day 0 (left paralumbar region) and day 7 (right paralumbar region) with an emulsion (125 μg per mouse) of myelin oligodendrocyte glycoprotein (mMOG-35-55 peptide) in CFA containing heat-killed Mycobacterium tuberculosis H37RA.

The 70 mice are randomized into 7 groups of 10 mice each. One group of 10 animals receives a saline injection to serve as an untreated EAE disease control. Six different formulations are evaluated in the remaining 6 groups. The mice of group 1 receive a placebo; group 2 receives rhAFP at 10 μg/day; group 3 receives rhAFP at 100 μg/day; group 4 receives IFN-β-1a at 0.1 μg/day; group 5 receives IFN-β-1a at 1 μg/day; group 6 receives both rhAFP and IFN-β-1a, at 10 μg and 0.1 μg respectively/day; and group 7 receives both rhAFP and IFN-β-1a, 100 μg and 1 μg respectively. The administration is by daily injections (ip or subcutaneous) from day 0 until the end of experiment at day 60. All groups are scored daily for disease symptoms according to the scale as described in Example 1 for the duration of the study.

At day 60, all mice are euthanized, and various organs and blood (e.g., spleen, knees, hind and fore paws) are harvested for immuno-histochemistry and immunological analysis.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference in their entirety.

What is claimed is:

1. A method of treating a patient with multiple sclerosis (MS), comprising administering to the patient a therapeutically effective amount of an alpha-fetoprotein (AFP) or a biologically active fragment, derivative, or analog thereof, and a therapeutically effective amount of an immunomodulatory agent.

2. The method of claim 1, wherein said AFP or biologically active fragment, derivative, or analog thereof is human AFP or a biologically active fragment, derivative, or analog thereof.

3. The method of claim 2, wherein said AFP or biologically active fragment, derivative, or analog thereof is non-glycosylated.

4. The method of claim 1, wherein said immunomodulatory agent is a protein.

5. The method of claim 4, wherein said protein is an interferon or glatiramer acetate.

6. (canceled)

7. The method of claim 1, wherein said immunomodulatory agent is an antibody.

8. (canceled)

9. The method of claim 1, wherein said immunomodulatory agent is BG12, fingolimod, mixoxantrone, laquinimod, teriflunomide, or atorvastatin.

10. (canceled)

11. The method of claim 1, wherein said AFP or biologically active fragment, derivative, or analog thereof, or said immunomodulatory agent, is administered daily, weekly, biweekly, or monthly.

12-21. (canceled)

22. The method of claim 1, wherein said AFP or said immunomodulatory agent is administered intravenously, intramuscularly, orally, by inhalation, parenterally, intraperitoneally, intraarterially, transdermally, sublingually, nasally, in a suppository, transbuccally, liposomally, adiposally, intraocularly, subcutaneously, intrathecally, topically, or through local administration.

23-24. (canceled)

25. The method of claim 1, further comprising administering one or more of a disease-modifying anti-rheumatic drug (DMARD), a corticosteroid, or a non-steroidal anti-inflammatory drug (NSAID) to said patient.

26-27. (canceled)

28. A composition comprising an alpha-fetoprotein (AFP) or biologically active fragment, derivative, or analog thereof, and an immunomodulatory agent.

29-35. (canceled)

36. The composition of claim 28, wherein said immunomodulatory agent is BG12, fingolimod, mixoxantrone, laquinimod, teriflunomide, or atorvastatin.

37. (canceled)

38. The composition of claim 28, which is formulated for intravenous, intramuscular, oral, by inhalation, parenteral, intraperitoneal, intraarterial, transdermal, sublingual, nasal, in a suppository, transbuccal, liposomal, adiposal, intraocular, subcutaneous, intrathecal, topical, or local administration.

39. The composition of claim 28, further comprising one or more of a disease-modifying anti-rheumatic agent (DMARD), a cortico steroid, or a non-steroidal anti-inflammatory drug (NSAID).

40. A kit comprising:
(i) a therapeutically effect amount of an alpha-fetoprotein (AFP) or a biologically active fragment, derivative, or analog thereof,
(ii) a therapeutically effective amount of an immunomodulatory agent; and
(iii) instructions for administering said AFP or biologically active fragment, derivative, or analog thereof and said immunomodulatory agent to a patient having multiple sclerosis.

41. The kit of claim 40, wherein said AFP or biologically active fragment, derivative, or analog thereof is human AFP or a biologically active fragment, derivative, or analog thereof.

42. The kit of claim 40, wherein said AFP or biologically active fragment, derivative, or analog thereof is non-glycosylated.

43. The kit of claim 40, wherein said immunomodulatory agent is a protein.

44-45. (canceled)

46. The kit of claim 40, wherein said immunomodulatory agent is an antibody.

47. (canceled)

48. The kit of claim 40, wherein said immunomodulatory agent is BG12, fingolimod, mixoxantrone, laquinimod, teriflunomide, or atorvastatin.

49. (canceled)

50. The kit of claim 40, wherein said AFP or biologically active fragment, derivative, or analog thereof, and/or said immunomodulatory agent is formulated for intravenous, intramuscular, oral, by inhalation, parenteral, intraperitoneal, intraarterial, transdermal, sublingual, nasal, in a suppository, transbuccal, liposomal, adiposal, intraocular, subcutaneous, intrathecal, topical, or local administration.

51. The kit of claim 40, wherein said AFP and said immunomodulatory agent are formulated for two different routes of administration or for the same route of administration.

52. (canceled)

53. The kit of claim 40, further comprising one or more of a disease-modifying anti-rheumatic agent (DMARD), a corticosteroid, or a non-steroidal anti-inflammatory drug (NSAID).

54-56. (canceled)
